(19) United States
(12) Patent Application Publication
(10) Pub. No.: US 2026/0169006 A1
(43) Pub. Date: Jun. 18, 2026

(54) CONFIRMATION OF PROTEIN IDENTIFICATION AND PURITY THROUGH COMPARABILITY

(71) Applicant: PROTEIN DYNAMIC SOLUTION, INC., Wakefield, MA (US)

(72) Inventor: Belinda PASTRANA-RIOS, Wakefield, MA (US)

(73) Assignee: PROTEIN DYNAMIC SOLUTION, INC., Wakefield, MA (US)

(21) Appl. No.: 19/535,584

(22) Filed: Feb. 10, 2026

Related U.S. Application Data
(63) Continuation of application No. PCT/US2024/041755, filed on Aug. 9, 2024.
(60) Provisional application No. 63/518,754, filed on Aug. 10, 2023.

(51) Int. Cl. G01N 33/68; G01N 21/552

(52) U.S. Cl. CPC G01N 33/6848; G01N 21/552

(57) ABSTRACT

The assessment of confirmation therapeutic protein identification and purity can be assessed through comparative analysis against a reference standard. Therapeutic protein identification is not only dependent on the amino acid sequence and its secondary structure, but also the post-translational modifications that it possesses and all impact its stability. Infrared microscopy is employed to acquire hyperspectral images of a fixed sample volume of the intact protein in solution. The spectral data obtained from these images are then analyzed by correlation algorithms, specifically: two trace in two-dimensions (2T2D) and two-dimensional correlation spectroscopy (2D-COS) providing a simple output for interpretation based on the 2T2D, and the dynamic fingerprint of the desired therapeutic protein using the 2D-COS. The effort is valid for both the initial evaluation of characterization and for follow-on assessment through the drug life cycle.

### CROSS-REFERENCE TO RELATED APPLICATIONS

This application is a continuation of International Application No. PCT/US2024/041755, filed on Aug. 9, 2024, which claims the benefit of U.S. Provisional Application No. 63/518,754, filed Aug. 10, 2023, the entirety of which are hereby incorporated by reference.

### STATEMENT REGARDING FEDERALLY SPONSORED RESEARCH

This invention was made with Government support under Award No. 1632420 awarded by the National Science Foundation. The Government has certain rights in this invention.

### FIELD OF THE INVENTION

The present invention relates to a system and method for rapid assessment of protein identification and purity through comparative analysis of acquired spectral data against a reference standard. In particular, the system and method acquire spectral images of solutions or mixtures of both a sample and a reference standard, and then analyze spectral data obtained from the spectral images using two-trace two-dimensional (2T2D) correlation analysis and two-dimensional correlation spectroscopy (2D-COS) to assess the identity of one or more proteins in the sample solution and any differences between the one or more proteins in the sample solution and corresponding reference standards of those proteins.

### BACKGROUND

The assessment of confirmation therapeutic protein identification and purity can be assessed through comparative analysis against a reference standard. Therapeutic protein identification is not only dependent on the amino acid sequence, its secondary structure, but also the post-translational modifications that it possesses and all impact its stability. Current methods of assessing identification and purity of proteins are labor intensive and require extensive sample preparation. In particular, current methods require long processing steps that may take days or even weeks to complete, depending on the extent of amino acid coverage to be determined. This can lead to an effective bottleneck with respect to the determination or confirmation of the identity of the protein.

What is needed is a system and method for reliably assessing the identification of a protein within a sample or mixture, and any differences between the identified protein and its corresponding reference standard.

### SUMMARY OF THE INVENTION

The system and method described herein provides a rapid method of assessing the identity of therapeutic proteins against one or more reference standards under controlled temperature conditions in a short time period, such as 10 minutes, to determine or confirm an identity of the proteins in a sample or mixture of proteins with the presence of: degradation products or impurities (such as deamidation or oxidization of the protein), aggregates, or variability in glycosylation of the protein which impacts its stability. This short timeframe is for the evaluation of a plurality of samples against a predetermined number of references, independent of size and modality. The reference standards may be reference samples of the protein, where the reference sample is of a known purity and may further be a reference sample of a known protein of a known purity vibrational spectroscopy, such as infrared spectroscopy which is highly selective and sensitive, may be used to evaluate the samples and a reference standard. In particular, after spectral images are obtained using vibrational spectroscopy, two-trace two-dimensional (2T2D) correlation spectroscopy can be applied to provide a comparative analysis between a protein sample against one or more reference standards at one or more controlled temperatures. This allows for the identity of the protein sample to be assessed.

Further, two-dimensional correlation spectroscopy (2D-COS) can be applied to the acquired spectral images in order to assess any differences between the sample protein and reference standard, such that a dynamic fingerprint, or record of the differences between the sample and reference standard, can be developed and assessed. The spectral comparisons described herein are valid for amino acid sequence, post-translational modification, oligomeric state, degradation, concentration and formulation conditions.

The system and method do not require performing long sample processing steps that require days to weeks depending on the extent of amino acid coverage to be determined, as is required by existing systems and methods for identifying proteins in a sample that result in an effective bottleneck towards the determination or confirmation of the protein ID. Rather, that the system and method described herein assess the dynamics of a protein sample when compared to one or more reference standards, and these dynamics which can include differences or changes in the sample protein as compared to the standard, are its fingerprint for identification during follow-on segments of development during the drug lifecycle. The system and method also allow for the identification of the components within a mixture when the results fail to confirm identity of the therapeutic protein against its reference standard. The method is robust because it evaluates the therapeutic proteins dynamics at different set temperatures. The method can also be applied to any size or type of protein, including, but not limited to, peptides, proteins, glycosylated proteins, antibodies, membrane associated proteins or engineered proteins, among others.

To evaluate samples, the system and method use a vibrational spectroscopy apparatus, such as a quantum cascade laser microscope (QCLM), Raman amplifier, FT-IR, or light detection and ranging (LIDAR) system, with a slide cell array and dedicated software that provide protein identification and evaluation. The slide cell array may be comprised of a polymer, such as polyethylene, calcium fluoride, barium fluoride, or other polymer that is transparent within most of the spectral region of interest. In embodiments, the samples to be screened, along with one or more reference standards, or controls, are provided within the cells of the slide cell array, and the QCLM is used to acquire spectral images of the slide cell array. The system may be implemented as a platform, with data from the QCLM provided to the dedicated software for analysis of the samples. The QCLMs provide real-time hyperspectral (HS) image acquisition with enhanced signal to noise ratio. The QCLM contains a light source, and the intensity of the light source may be comparable to the synchrotron and is non-destructive. Each hyperspectral image obtained is comprised of 223,000 spectra. The hyperspectral images may be acquired within the spectral region of 1800-1480 cm-1 at a spectral resolution of 4 cm-1. Although the spectral region of interest may include the full spectral region 1800-950 cm-land the spectral resolution may be 2 cm-1 to access post-translational modifications (PTM) and formulation information. As mentioned, the hyperspectral images may also be acquired by other vibrational spectroscopy apparatus, such as a Raman amplifier, Raman microscope, or LIDAR. The spectral images may then be analyzed to determine the identity and purity of the sample proteins.

In particular, a comparative analysis of the protein sample against its reference standard at multiple temperatures is performed to provide a robust assessment of the identity and purity of the therapeutic protein. The analysis process implements 2T2D correlation spectroscopy that provides weighted spectral difference for the sample and the reference within the spectral region of interest. When the protein sample is identical, or nearly identical, to that of the reference standard, then weighted difference spectra for the sample and reference will be zero or within a predetermined threshold of zero, confirming that the identity of the sample is the reference protein. This may result in, for example, a weighted difference spectra represented by a line having a zero or near zero slope. Where the acquired the spectral data are for a sample containing a mixture of proteins or one or proteins and their degradation products, the components of the mixture can be identified using the 2T2D spectroscopic method. In particular, by analyzing weighted difference spectra of the sample, contaminants or degraded product can be identified by interpreting the spectra and evaluating the amide I and II bands for differences in conformation side chain composition and weak interactions. For example, the a Euclidian distance value can be calculated between the weighted difference spectra, with a Euclidian distance value of zero indicating that the protein in the sample is the same as the reference protein. The Euclidian distance may be calculated by comparing intensity values of the weighted reference against the weighted sample along the spectra, with the Euclidian distance equation giving a total value. For example, weighted spectral subtraction, may be performed with a constant weight to yield the smallest nonzero result:

$$p ( v ) = s ( v ) - kr \cdot r ( v ) \geq 0$$

$$q ( v ) = r ( v ) - ks \cdot s ( v ) \geq 0$$

where kᵣ and kₛ are iteratively estimated. The weighted difference spectrum obtained by this spectral subtraction with the non-negativity constraint allows for the estimation of the contribution of the components within a sample when compared to the reference spectrum. The Euclidian distance is then determined for the weighted difference spectra to determine spectral similarity between the reference and sample:

$$d ( p ( v ) , q ( v ) ) = \sqrt{{( p ( {v}_{1} ) - q ( {v}_{1} ) )}^{2} + {( p ( {v}_{2} ) - q ( {v}_{2} ) )}^{2} + … + {( p ( {v}_{n} ) - q ( {v}_{n} ) )}^{2}}$$

where p is weighted intensity difference at v wavenumber for the reference and q is the corresponding weighted intensity difference at v wavenumber for the sample. The determination is performed for all the spectral data points within the spectral region of interest.

Linear regression may also be used to analyze the weighted difference spectra, with a resulting line having a slope of zero again confirming the identity of the sample protein as that of the reference protein. In addition, 2T2D synchronous and asynchronous plots may be generated by correlating sample spectral data and reference standard spectral data, and the presence of cross peaks in the asynchronous plot indicate differences between the sample and reference standard.

Contaminants or components of mixtures of proteins may also be identified by using multiple known reference standard samples. By comparing the sample data to the data for each of the known reference standard samples, contaminants and components can be confirmed.

The method further comprises applying two-dimensional correlation spectroscopy (2D-COS) to enable assessment of differentiation of the therapeutic proteins as compared to the reference standard, with these differences being part of a dynamic fingerprint of the sample. IR is highly sensitive and selective, yet when coupled to 2D-COS analysis there is enhanced spectral and temporal resolution allowing for a comprehensive biochemical comparison between the sample and its reference. The 2D-COS assessment may include generation of synchronous and asynchronous plots from the spectral data, and evaluation of peaks within these plots. For example, purity of a protein during thermal perturbation may be assessed by evaluating the number and position of the cross peaks within the 2D-COS asynchronous plots for a spectral data set acquired at three or more different set temperatures.

As a result, a comprehensive evaluation that includes multiple elements: (1) differences in amino acid sequence, (2) secondary structure contributions, (3) the weak and covalent interactions that govern secondary structure, (4) post-translational modifications such as extent of glycosylation, degradation and payload, (5) stability (6) determination of the presence of a mixture of proteins being present in a sample and (7) key signature peak associated with the excipient when compared to a reference standard can be performed.

### Protein Identification and Assessment

For example, a method for performing protein identification screening of a sample may comprise:
a) providing the sample in a slide containing at least one sample well and at least one reference well;
b) acquiring at least one spectral image of the sample and at least one spectral image of the at least one reference using a quantum cascade laser microscope under controlled temperature conditions;
c) identifying and selecting, in at least one of the acquired first spectral images, a region of interest;
d) obtaining spectral data for the sample and the at least one reference for the region of interest;
e) applying a baseline correction to the spectral data for the sample and the reference for the region of interest;
f) applying a two-trace two-dimensional correlation to the baseline corrected sample and reference spectral data to generate a synchronous spectrum Φ (v₁, v₂) and asynchronous spectrum Ψ(v₁, v₂);
g) generating weighted difference spectra for the sample and reference; and
h) analyzing the weighted difference spectra and confirming an identity of the sample protein when the weighted difference is within a predetermined threshold.

In the method, acquiring at least one spectral image of the sample using a quantum cascade laser microscope includes acquiring at least one first hyperspectral image of the sample.

The method may further comprise applying a 2T2D algorithm to generate synchronous and asynchronous plots:

$$Φ ( {v}_{1} , {v}_{2} ) = \frac{1}{2} [ s ( {v}_{1} ) \cdot s ( {v}_{2} ) + r ( {v}_{1} ) \cdot r ( {v}_{2} ) ]$$

$$Ψ ( {v}_{1} , {v}_{2} ) = \frac{1}{2} [ s ( {v}_{1} ) \cdot r ( {v}_{2} ) - r ( {v}_{1} ) \cdot s ( {v}_{2} ) ]$$

where Φ(u₁, u₂) is the synchronous plot (also referred to herein as the synchronous spectrum) and Ψ(u₁, u₂) is the asynchronous.

The method may further include applying a 2T2D correlation coefficient ρ(v₁, v₂) to the synchronous spectrum, and applying a disrelation coefficient ξ(v₁, v₂) to the asynchronous spectrum, as given by:

$$ρ ( {v}_{1} , {v}_{2} ) = Φ ( {v}_{1} , {v}_{2} ) / \sqrt{Φ ( {v}_{1} , {v}_{1} ) \cdot Φ ( {v}_{2} , {v}_{2} )}$$

$$ξ ( {v}_{1} , {v}_{2} ) = Ψ ( {v}_{1} , {v}_{2} ) / \sqrt{Φ ( {v}_{1} , {v}_{1} ) \cdot Φ ( {v}_{2} , {v}_{2} )}$$

$$where :$$

$${ρ ( {v}_{1} , {v}_{2} )}^{2} + {ξ ( {v}_{1} , {v}_{2} )}^{2} = 1 .$$

The method may further include applying 2D-COS correlation analysis to the spectral data, and analyzing cross peaks in generated synchronous and asynchronous plots to determine the dynamics of the protein.

### BRIEF DESCRIPTION OF THE DRAWINGS

FIG. 1 is an illustration of the amino acid sequence for two commercial insulins, Humulin R and Humalog, respectively.

FIG. 2A is an illustration of a spectral overlay within the spectral region of 1740-1500 cm⁻¹ within a temperature range of 26-42° C., from spectral images acquired with a QCLM at temperature intervals of 4° C. and 4-minute equilibrium periods for Humulin R.

FIG. 2B is an illustration of a spectral overlay within the spectral region of 1740-1500 cm⁻¹ within a temperature range of 26-42° C., from spectral data acquired with a QCLM at temperature intervals of 4° C. and 4-minute equilibrium periods for Humalog.

FIG. 3A is an illustration of a spectral overlay for a sample being analyzed to determine if it is Humulin R and a reference sample of Humulin R at room temperature within the spectral region of 1740-1500 cm⁻¹ from spectral data acquired with a QCLM.

FIG. 3B is an illustration of a 2T2D synchronous plot generated by applying a 2T2D correlation to the pair of spectra for the Humulin R sample and Humulin R reference sample illustrated in FIG. 3A.

FIG. 3C is an illustration of a 2T2D asynchronous plot generated by applying a 2T2D correlation to the pair of spectra for the Humulin R sample and Humulin R reference sample illustrated in FIG. 3A.

FIG. 4A is an illustration of a 2T2D weighted difference spectra for the reference sample of Humulin R.

FIG. 4B is an illustration of a 2T2D weighted difference spectra for the sample of Humulin R.

FIG. 5A is an illustration of a spectral overlay for a known sample of Humulin R and a known sample of Humalog within the spectral region of 1740-1500 cm⁻¹ from spectral data acquired with a QCLM.

FIG. 5B is an illustration of a 2T2D synchronous plot generated by applying a 2T2D correlation to the pair of spectra for the known sample of Humulin R and a known sample of Humalog illustrated in FIG. 5A.

FIG. 5C is an illustration of a 2T2D asynchronous plot generated by applying a 2T2D correlation to the pair of spectra for the known sample of Humulin R and a known sample of Humalog illustrated in FIG. 5A.

FIG. 6A is an illustration of a 2T2D weighted difference spectra for the known sample of Humulin R.

FIG. 6B is an illustration of a 2T2D weighted difference spectra for the known sample of Humalog.

FIG. 7A is an illustration of a spectral overlay for a sample being analyzed to determine if it is Humalog and a reference sample of Humalog within the spectral region of 1740-1500 cm⁻¹ from spectral data acquired with a QCML.

FIG. 7B is an illustration of a 2T2D synchronous plot generated by applying a 2T2D correlation to the pair of spectra for the Humalog sample and Humalog reference sample illustrated in FIG. 7A.

FIG. 7C is an illustration of a 2T2D asynchronous plot generated by applying a 2T2D correlation to the pair of spectra for the Humalog sample and Humalog reference sample illustrated in FIG. 7A.

FIG. 8A is an illustration of a 2T2D weighted difference spectra for the reference sample of Humalog.

FIG. 8B is an illustration of a 2T2D weighted difference spectra for the sample of Humalog.

FIG. 9A shows a 2D-COS synchronous plot for a Humulin R sample within the spectral region of 1740-1500 cm⁻¹, generated from spectral data acquired for the temperature range of 26-42° C. with the spectral data acquired at temperature intervals of 4° C.

FIG. 9B shows a 2D-COS asynchronous plot for a Humulin R sample within the spectral region of 1740-1500 cm⁻¹, generated from spectral data acquired for the temperature range of 26-42° C. with the spectral data acquired at temperature intervals of 4° C.

FIG. 10A shows a 2D-COS synchronous plot for a Humalog sample within the spectral region of 1740-1500 cm⁻¹, generated from spectral data acquired for the temperature range of 26-42° C. with the spectral data acquired at temperature intervals of 4° C.

FIG. 10B shows a 2D-COS asynchronous plot for a Humulin R sample within the spectral region of 1740-1500 cm⁻¹, generated from spectral data acquired for the temperature range of 26-42° C. with the spectral data acquired at temperature intervals of 4° C.

FIG. 11A shows an illustration of a spectral overlay for a known sample of a full-length engineered monoclonal antibody designated 1 (mAb1) and a reference sample of mAb1 at room temperature within the spectral region 1780-1015 cm⁻¹ acquired using a QCLM.

FIG. 11B is an illustration of a 2T2D weighted difference spectra for the reference sample of mAb1 within the full spectral region of 1780-1015 cm⁻¹.

FIG. 11C is an illustration of a 2T2D weighted difference spectra for the sample of mAb1 within the full spectral region of 1780-1015 cm⁻¹.

FIG. 11D is an illustration of the 2T2D asynchronous plot which is featureless due to the identity of the mAb1 sample when compared to the mAb1 reference. The asynchronous plot with a color bar scale at 1×10⁻¹⁵ A.U.

FIG. 11E shows an illustration of a spectral overlay of a mAb1 and a reference sample of mAb1 at room temperature within the spectral region 1780-1486 cm⁻¹.

FIG. 11F is an illustration of a 2T2D weighted difference spectra for the reference sample of mAb1 within the amide I and II band spectral region of 1780-1486 cm⁻¹.

FIG. 11G is an illustration of a 2T2D weighted difference spectra for the sample of mAb1 within the spectral region of 1780-1486 cm⁻¹.

FIG. 12A shows an illustration of the spectral overlay of a glycosylated extracellular domain target sample and a reference sample of the glycosylated target at room temperature within the spectral region 1780-1015 cm⁻¹ acquired using a QCLM.

FIG. 12B is an illustration of a 2T2D weighted difference spectra for the reference sample of a glycosylated target within the spectral region of 1780-1015 cm⁻¹.

FIG. 12C is an illustration of a 2T2D weighted difference spectra for the sample of a glycosylated target within the spectral region of 1780-1015 cm⁻¹.

FIG. 12D shows an illustration of the spectral overlay of a glycosylated extracellular domain target and a reference sample of the glycosylated target at room temperature within the amide I and II band spectral region 1780-1485 cm⁻¹ acquired using a QCLM.

FIG. 12E is an illustration of a 2T2D weighted difference spectra for the reference sample of a glycosylated target within the spectral region of 1780-1485 cm⁻¹.

FIG. 12F is an illustration of a 2T2D weighted difference spectra for the sample of a glycosylated target within the spectral region of 1780-1485 cm⁻¹.

FIG. 13A shows an illustration of the spectral overlay of a mAb1 sample and a reference sample of the glycosylated target at room temperature within the spectral region 1780-1015 cm⁻¹ acquired using a QCLM.

FIG. 13B is an illustration of a 2T2D weighted difference spectra for the reference sample of a glycosylated target within the spectral region of 1780-1015 cm⁻¹.

FIG. 13C is an illustration of a 2T2D weighted difference spectra for the sample of mAb1 within the full spectral region of 1780-1015 cm⁻¹ FIG. 13D is an illustration of the 2T2D asynchronous plot of the mAb1 sample when compared to the glycosylated target reference sample within the full spectral region of 1780-1015 cm⁻¹.

FIG. 13E shows an illustration of the spectral overlay of a mAb1 sample and a reference sample of the glycosylated target at room temperature within the spectral region 1780-1486 cm⁻¹ acquired using a QCLM.

FIG. 13F is an illustration of a 2T2D weighted difference spectra for the reference sample of a glycosylated target within the spectral region of 1780-1486 cm⁻¹.

FIG. 13G is an illustration of a 2T2D weighted difference spectra for the sample of mAb1 within the amide I and II band spectral region of 1780-1486 cm⁻¹.

FIG. 13H is an illustration of the 2T2D asynchronous plot of the mAb1 sample when compared to the glycosylated target reference sample within the spectral region of 1780-1486 cm⁻¹.

FIG. 13I shows a 2D-COS synchronous plot for a mAb1 sample within the spectral region of 1780-1486 cm⁻¹, generated from spectral data acquired for the temperature range of 24-52° C. with the spectral data acquired at temperature intervals of 4° C.

FIG. 13J shows a 2D-COS asynchronous plot for a mAb1 sample within the spectral region of 1780-1486 cm⁻¹, generated from spectral data acquired for the temperature range of 24-52° C. with the spectral data acquired at temperature intervals of 4° C.

FIG. 13K shows a 2D-COS synchronous plot for a glycosylated target sample within the spectral region of 1780-1486 cm⁻¹, generated from spectral data acquired for the temperature range of 24-52° C. with the spectral data acquired at temperature intervals of 4° C.

FIG. 13L shows a 2D-COS asynchronous plot for a glycosylated target sample within the spectral region of 1780-1486 cm⁻¹, generated from spectral data acquired for the temperature range of 24-52° C. with the spectral data acquired at temperature intervals of 4° C.

FIG. 14A shows an illustration of the spectral overlay of a glycosylated target/mAb2 (2.0/1.0, mol ratio) mixture sample and a reference sample of the pure glycosylated target at room temperature within the spectral region 1780-1015 cm⁻¹ acquired using a QCLM.

FIG. 14B is an illustration of a 2T2D weighted difference spectra for the reference sample of a glycosylated target within the spectral region of 1780-1015 cm⁻¹.

FIG. 14C is an illustration of a 2T2D weighted difference spectra for the sample of glycosylated target/mAb2 (2.0/1.0, mol ratio) mixture within the full spectral region of 1780-1015 cm⁻¹.

FIG. 14D is an illustration of the 2T2D asynchronous plot of the glycosylated target/mAb2 (2.0/1.0, mol ratio) mixture sample and a reference sample of the pure glycosylated target within the full spectral region of 1780-1015 cm⁻¹.

FIG. 14E shows an illustration of the spectral overlay of a glycosylated target/mAb2 (2.0/1.0, mol ratio) mixture sample and a reference sample of the pure glycosylated target at room temperature within the spectral region 1780-1486 cm⁻¹ acquired using a QCLM.

FIG. 14F is an illustration of a 2T2D weighted difference spectra for the reference sample of a glycosylated target within the amide I and II band spectral region of 1780-1486 cm⁻¹.

FIG. 14G is an illustration of a 2T2D weighted difference spectra for the sample of glycosylated target/mAb2 (2.0/1.0, mol ratio) mixture within the spectral region of 1780-1486 cm⁻¹.

FIG. 14H is an illustration of the 2T2D asynchronous plot of the glycosylated target/mAb2 (2.0/1.0, mol ratio) mixture sample and a reference sample of the pure glycosylated target within the amide I and II band spectral region of 1780-1486 cm⁻¹.

FIG. 14I shows a 2D-COS synchronous plot for a glycosylated target/mAb2 (2.0/1.0, mol ratio) mixture sample within the spectral region of 1780-1486 cm⁻¹, generated from spectral data acquired for the temperature range of 24-52° C. with the spectral data acquired at temperature intervals of 4° C.

FIG. 14J shows a 2D-COS asynchronous plot for a glycosylated target/mAb2 (2.0/1.0, mol ratio) mixture sample within the spectral region of 1780-1486 cm⁻¹, generated from spectral data acquired for the temperature range of 24-52° C. with the spectral data acquired at temperature intervals of 4° C.

FIG. 15A shows an illustration of a mAb2 sample and a reference sample of mAb1 at room temperature within the spectral region 1780-1015 cm⁻¹ acquired using a QCLM.

FIG. 15B is an illustration of a 2T2D weighted difference spectra for the reference sample of mAb1 within the full spectral region of 1780-1015 cm⁻¹.

FIG. 15C is an illustration of a 2T2D weighted difference spectra for the sample of mAb2 within the full spectral region of 1780-1015 cm⁻¹.

FIG. 15D is an illustration of the 2T2D asynchronous plot of the mAb2 sample when compared to the mAb1 reference full spectral region of 1780-1015 cm⁻¹.

FIG. 15E shows an illustration of a spectral overlay of a mAb2 sample and a reference sample of mAb1 at room temperature within the spectral region 1780-1486 cm⁻¹.

FIG. 15F is an illustration of a 2T2D weighted difference spectra for the reference sample of mAb1 within the amide I and II band spectral region of 1780-1486 cm⁻¹.

FIG. 15G is an illustration of a 2T2D weighted difference spectra for the sample of mAb2 within the spectral region of 1780-1486 cm⁻¹.

FIG. 15H is an illustration of the 2T2D asynchronous plot of the mAb2 sample when compared to the mAb1 reference within the amide I and II band spectral region of 1780-1486 cm⁻¹.

FIG. 16 is an illustration of flow diagrams for the processes of assessing protein sample identity and purification against a reference standard.

### DETAILED DESCRIPTION

The system and method described herein provide for a rapid process for identifying and assessing a sample protein by comparison against a reference standard of the sample.

### Sample Preparation

The system and method subject a series of therapeutic protein samples in their formulations to a comparative analysis with one or more reference standards at one or more controlled temperatures. The method is not limited by the therapeutic protein entity or modality. The protein concentration range is optimized for the analysis, with both the reference protein and sample being in the same formulation condition. This effectively minimizes sample preparation steps. A slide cell array may be provided that includes a plurality of wells. The slide cell array may be comprised of a salt, a polymer, such as polyethylene, calcium fluoride, barium fluoride, or other polymer that is transparent within most of the spectral region of interest. Samples to be assessed and reference samples are placed in pre-defined wells within the slide array. Only a small amount of a sample, such as 1 μL, sample per well, is required for analysis. After the samples and reference samples are placed in the slide, the slide cell is then covered and assembled into a thermally controlled slide cell holder to ensure a controlled thermal environment. The cover may be an optically polished cover. The wells may be a pre-determined depth, and the cover may be a predetermined thickness, thereby providing a fixed path-length for assessment. The slide cell holder accessory may be, for example, a controllable heated chamber configured to receive the slide cell.

### Hyperspectral Image Acquisition

After the slide cell array is placed in the slide cell holder, spectral images of the slide cell array are obtained. These spectral images may be obtained using real-time hyperspectral (HS) image acquisition by a Quantum Cascade Laser Microscope (QCLM). The QCLM HS image acquisition may be carried out for all protein samples and reference samples within the slide cell array at a one or more defined temperatures. For example, the HS image acquisition may be conducted at a defined temperature of 25±0.3° C. The HS images may be acquired within a spectral region of interest from 1775-1435 cm⁻¹, which includes both the amide I and amide II bands. The spectral region may also be expanded to include other ranges, such as 1800-1480 cm⁻¹ or 1800-950 cm⁻¹. HS images may be acquired within a desired temperature range, with predetermined equilibration periods in between acquiring HS images at different temperatures within the range. For example, the equilibration periods may be 4 minutes. Each HS image acquisition occurs within seconds, and may be comprised of 223,000 QCLM spectra collected at 4 cm⁻¹ spectral resolution within a desired spectral region, such as the mid-IR spectral region. The HS images can also be acquired for broader spectral regions, such as 1800-950 cm⁻¹, or narrower spectral regions, if desired. Other microscopy techniques may be applied to obtain the spectral images, such as FT-IR or Raman microscopy. The enhanced signal to noise ratio, real-time HS image acquisition and non-destructive nature of the light source obtained using the QCLM enable the small sample requirements that benefit the biopharma industry in sample assessment.

### 2T2D Correlation Analysis

After the spectral images are acquired, a two-trace two-dimensional (2T2D) correlation¹ process is applied to the spectral data. From a pair of spectra a two trace two-dimensional correlation can be applied to generate a synchronous spectrum Φ(v₁, v₂) and asynchronous spectrum Ψ(v₁, v₂). The synchronous spectrum Φ(v₁, v₂) and asynchronous spectrum Ψ(v₁, v₂) are given by:

$$Φ ( {v}_{1} , {v}_{2} ) = \frac{1}{2} [ s ( {v}_{1} ) \cdot s ( {v}_{2} ) + r ( {v}_{1} ) \cdot r ( {v}_{2} ) ] \qquad ( 1 )$$

$$Ψ ( {v}_{1} , {v}_{2} ) = \frac{1}{2} [ s ( {v}_{1} ) \cdot r ( {v}_{2} ) - r ( {v}_{1} ) \cdot s ( {v}_{2} ) ] \qquad ( 2 )$$

The first, original spectra, s (v), corresponds to the sample and the second spectra, r(v), corresponds to the reference, respectively. A 2T2D correlation coefficient is then applied ρ(v₁, v₂) to the synchronous evaluation, and a disrelation coefficient ξ(v₁, v₂) to the asynchronous evaluation resulting in the scaled version of the 2T2D correlation spectra, as given by:

$$ρ ( {v}_{1} , {v}_{2} ) = Φ ( {v}_{1} , {v}_{2} ) / \sqrt{Φ ( {v}_{1} , {v}_{1} ) \cdot Φ ( {v}_{2} , {v}_{2} )} \qquad ( 3 )$$

$$ξ ( {v}_{1} , {v}_{2} ) = Ψ ( {v}_{1} , {v}_{2} ) / \sqrt{Φ ( {v}_{1} , {v}_{1} ) \cdot Φ ( {v}_{2} , {v}_{2} )} \qquad ( 4 )$$

$$Where :$$

$${ρ ( {v}_{1} , {v}_{2} )}^{2} + {ξ ( {v}_{1} , {v}_{2} )}^{2} = 1 \qquad ( 5 )$$

This indicates the complementarity nature of the quantities.

Two contour plots are generated. These are the synchronous (Φ(v₁, v₂)) plot where dominant spectral components of the samples: s(v) and r(v) are observed. The diagonal is comprised of auto peaks where v₁=v₂. The cross peaks are always positive. The second plot is the asynchronous (Ψ(v₁, v₂)) plot, which is more informative. In the case Ψ>0, then the intensity contribution of the functional group is from vi, corresponding to the first component being more abundant. In the case Ψ<0, then the intensity contribution of the functional group is from v₂, therefore, the second component of the sample is more abundant. Also, this indicates that peaks of the same intensity and sign correspond to the same component within the sample.

### 2D-COS Analysis

2D-COS has proven useful in that it provides a detailed molecular description of the effects of a perturbation on both the side chains and, in turn, the conformational stability of a protein. The stability of a protein is dependent on its amino acid sequence, secondary and tertiary structure, post-translational modifications and formulation conditions. This correlation analysis enhances the spectral resolution to provide such information as the protein is destabilized, therefore serving as a true fingerprint for its identification. The 2D-COS algorithm is defined as:

$$\tilde{A} ( {v}_{j} , {t}_{k} ) = \{ \begin{matrix}A ( {v}_{j} , {t}_{k} ) - \overline{A} ( {v}_{j} ) & if 1 \leq k \leq m \\ 0 & otherwise\end{matrix} \qquad ( 1 )$$

where, Ā(vⱼ) is the initial spectrum of the dataset.

Synchronous 2D correlation intensities of the covariance spectral data are defined by:

$$Φ ( {v}_{1} , {v}_{2} ) = \tilde{A} ( {v}_{1} , {t}_{j} ) \cdot \tilde{A} ( {v}_{2} , {t}_{j} ) \qquad ( 2 )$$

The resulting correlation intensity Φ(v₁, v₂) as a function of two independent wavenumber axes, v₁ and v₂, is the synchronous plot.

Asynchronous 2D correlation intensities of the covariance spectral data are defined by:

$$Ψ ( {v}_{1} , {v}_{2} ) = \tilde{A} ( {v}_{1} , {t}_{j} ) \cdot {N}_{ij} \tilde{A} ( {v}_{2} , {t}_{i} ) \qquad ( 3 )$$

The term Nᵢⱼ is the element of the so-called Hilbert-Noda transformation matrix and is given by:

$${N}_{ij} = \{ \begin{matrix}0 & for i = j \\ \frac{1}{π ( j - i )} & otherwise\end{matrix} \qquad ( 4 )$$

The cross-correlation function is applied to a difference spectral dataset, which is a dataset obtained from subtracting an initial spectrum from subsequent spectra. For example, the initial spectrum may be acquired at low temperature, and subsequent spectra acquired at higher temperatures. The spectral changes due to temperature increase are then revelated (revealing changes in the protein behavior), which are referred to as covariance spectral data or difference spectra. Applying the cross-correlation function to the different spectral dataset results in two separate, yet symmetrical 2D plots. The first plot is referred to as the synchronous plot. It contains positive peaks on the diagonal, known as the auto peaks, and provides the overall changes observed in the spectral dataset. The relationship established in this synchronous plot relates to the spectral intensity changes that occur synchronously, hence the name. The second 2D plot is known as the asynchronous plot. This plot relates the asynchronous intensity changes, resulting in enhanced spectral resolution, which can be used to correlate out-of-phase peak intensity changes, such as those defined for the deamidation process.

Both plots contain off-diagonal peaks, which are referred to as cross-peaks; these peaks correlate the spectral changes observed at the frequencies assigned to the various bands (e.g., backbone and side chain structural elements). Spectral intensity changes observed are due to the incremental thermal perturbation applied to the protein sample. No a priori knowledge of the system is required for interpretation of the results. The information in both plots allows for determining the sequential order of molecular events that occur during the perturbation by following Noda's rules. These plots are symmetrical in nature and for interpretation purposes, reference is made to the top triangular portion of the plot for analysis. The asynchronous plot is comprised exclusively of cross peaks that relate the out-of-phase peaks. As a result this plot reveals greater spectral resolution enhancement. The following rules can apply to establish the order of molecular events:
I. If the asynchronous cross peak v₂: if positive, then v₂ is perturbed prior to v₁ (v₂→v₁).
II. If the asynchronous cross peak v₂: if negative, then v₂ is perturbed after v₁ (v₂←v₁).
III. If the corresponding synchronous cross peak is positive, then the order of the events is established using the asynchronous plot (rules I and II).
IV. However, if the corresponding synchronous cross peak is negative and the asynchronous cross peak is positive, the order is reversed.
V. If the synchronous plot contains negative cross peaks and the corresponding asynchronous cross peak is negative, then the order is maintained.

An order of events can be established for each peak observed on the v₂ axis.

Moreover, by comparing the position of the peaks in the synchronous and asynchronous plots to the expected positions of such peaks for synchronous and asynchronous plots of a reference sample, differences between the sample and the reference standard can be assessed. These differences may be due to, for example, the amino acid sequence, post-translational modifications, excipient presence, perturbation and formulation conditions. The differences provide information on the purity of the sample.

### Protein Identification and Purity Assessment

The combined assessment of the protein samples and reference samples using the 2T2D and 2D-COS algorithms provides an in-depth assessment for therapeutic protein authentication: (1) a rapid comparability assessment to evaluate protein ID using the 2T2D algorithm and (2) a more dynamic fingerprint that is sensitive to the amino acid sequence, post-translational modifications, excipient presence, perturbation and formulation conditions. That is, by generating weighted difference spectra between the sample and the reference protein using 2T2D, the different spectra can be compared to a predetermined threshold. Where the difference is within the threshold, which may be close to zero, then the identity of the sample protein is confirmed as being the same as the reference sample. Applying 2D-COS provides for dynamic evaluation of any differences between the sample proteins and the reference protein samples. For example, the locations of peaks within the 2D-COS synchronous and asynchronous plots and the correlations established allow for comparability assessment. The direct comparison of the 2D-COS plots to where expected peaks would be for a reference sample, provide information on the particular characteristics and purity of the sample being analyzed. Information on relative stability may also be assessed by evaluating the sequential order of events derived from the analysis of the plots.

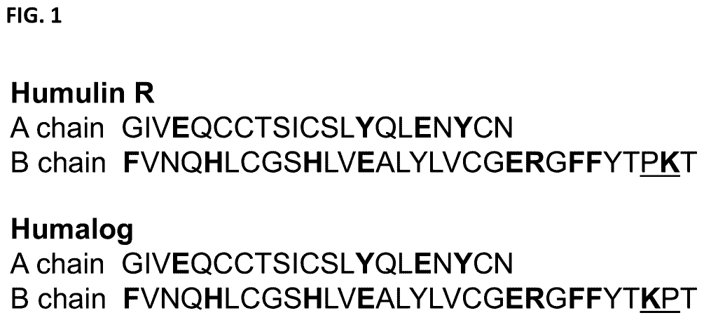

FIG. 1 provides an illustration of the amino acid sequence for two known commercial insulins: Humulin R shown at the top of FIG. 1 and Humalog shown at the bottom. Both insulins contain 3 disulfide bonds. The difference between the two is limited to chain B, a 30-residue peptide where the position of two amino acids at the C-terminal end has been switched. For Humulin R it is Pro28 and Lys29, while for Humalog it is Lys29 and Pro28. Also, Humulin R forms a hexamer and binds to zinc. The system and method described herein can distinguish between the two commercial insulins. Moreover, while the detailed discussion herein refers to Humulin R and Humalog as the reference proteins, the system and method can be applied using any known reference protein to which protein samples can be compared.

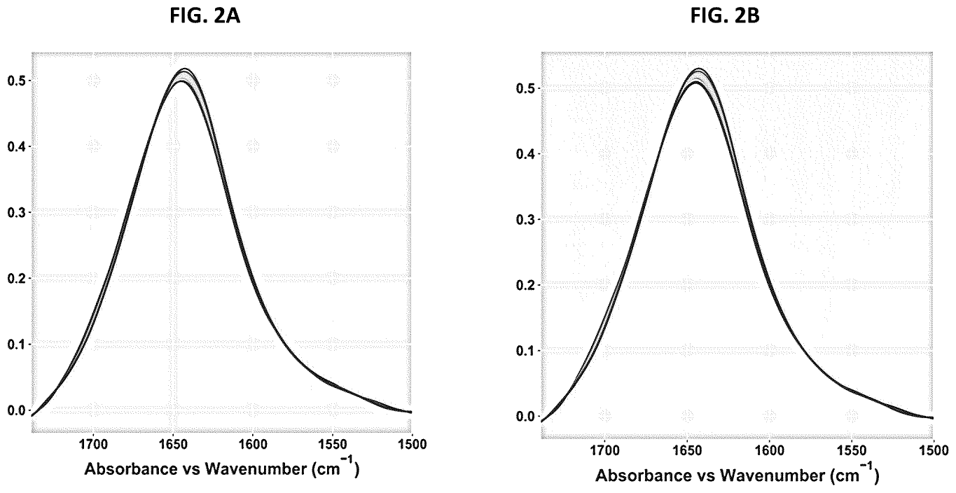

FIGS. 2A-B provide an illustration of QCLM spectral image overlays within the spectral region of 1740-1500 cm⁻¹ for Humulin R and Humalog, respectively, in their formulation conditions. The spectral images were acquired within a temperature range of 26-42° C., at temperature intervals of 4° C. and with 4-minute equilibrium periods. As can be seen, just comparing the spectral overlays to each other does not provide sufficient spectral resolution to distinguish the two insulins from each other.

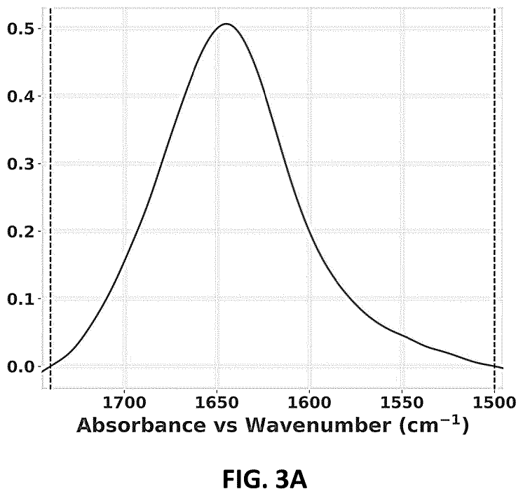

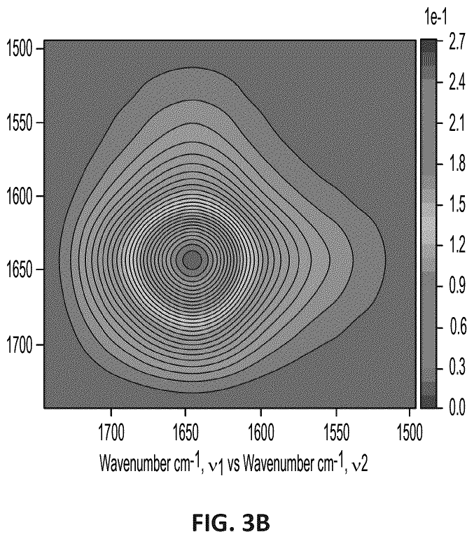

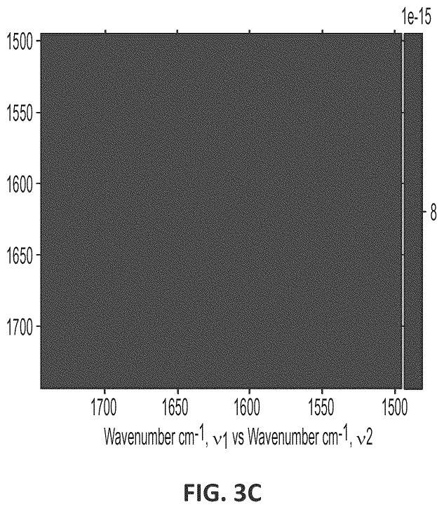

FIGS. 3A-C provide an illustration of a Humulin R protein identification spectral evaluation within the spectral region of 1740-1500 cm⁻¹ using the protein identity and purity assessment process. This protein identification evaluation includes generating a QCLM spectral overlay of spectral data obtained for two samples of Humulin R at room temperature-one of which can be considered a protein sample and the other a known reference sample of Humulin R. FIG. 3A illustrates the spectral overlay of the protein sample and reference sample of Humulin R at room temperature, with the overlay showing absorbance vs wavenumber. As described above, a 2T2D correlation algorithm is applied, which compares the spectra of the sample being evaluated to the spectra of the reference standard sample. The 2T2D correlation process may be applied to the acquired spectral data to generate a 2T2D synchronous spectrum and a 2T2D asynchronous spectrum. Two contour plots may then be generated from the synchronous and asynchronous spectra—a synchronous plot and an asynchronous plot. FIG. 3B illustrates an example of a 2T2D synchronous plot generated from the spectra data of the sample and reference standard. FIG. 3C illustrates an example of a 2T2D asynchronous plot generated from the spectra data of the sample and reference standard. The resulting 2T2D asynchronous plot shown in FIG. 3C, with no contours or peaks resulting from differences between the sample and reference standard spectra, is a typical result where the sample is identical to the reference standard. Where the sample is not the same as the reference standard, for example where the sample is a different protein than the reference standard protein, then the asynchronous plot would include contours and peaks indicative of the two not being the same.

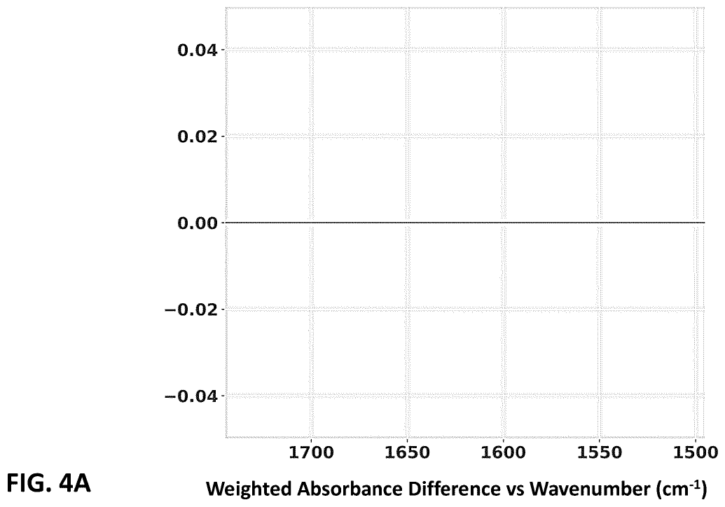

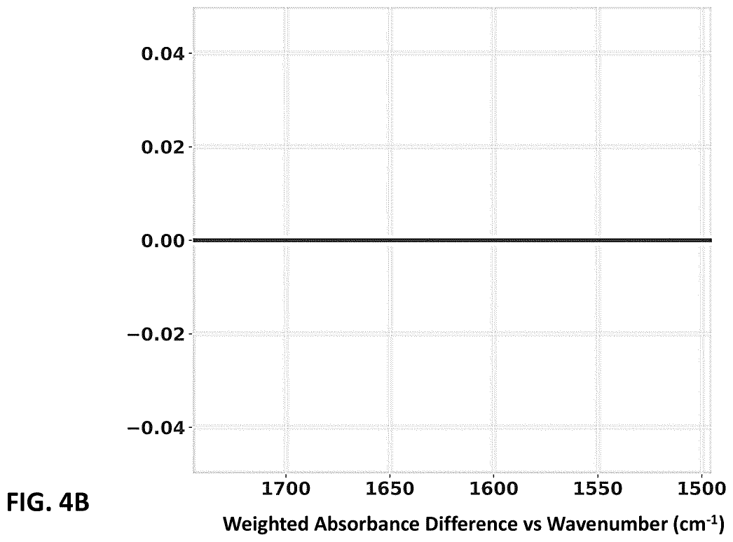

The method and system may further include generating 2T2D weighted difference spectra for the sample being evaluated and the reference standard sample. Such 2T2D weighted difference spectra are generated by subjecting the spectra to a weighted scaling factor that is defined for each: sample and reference spectrum, which will yield the smallest nonzero result. This treatment allows for the determination of the minor and major components within a mixture. This allows for the FIG. 4A illustrates the 2T2D weighted difference spectra for the Humulin R reference standard sample, and FIG. 4B illustrates the 2T2D weighted difference spectra for the sample being analyzed. The weighted difference spectra were generated from data acquired for both the reference standard sample and sample being analyzed at room temperature. Where the weighted difference is within a predetermined threshold, a positive identification of the protein is confirmed. As shown, both FIG. 4A and FIG. 4B show a single line with a slope of zero or near zero value. This indicates that there is no difference between the sample being analyzed and the Humulin R reference standard sample, confirming the identity of the sample being analyzed as Humulin R. That is, when the weighted difference spectra shows only a difference between the sample being analyzed and the reference standard sample that is zero or near zero, then the sample being analyzed can be identified as being the same protein as the reference standard sample. Moreover, a Euclidian distance calculation between the weighted difference spectra of FIG. 4A as compared to the weighted difference spectra of FIG. 4B would be zero, indicating the two spectra are identical. This would therefore confirm the identity of the therapeutic protein sample as being the same as that of the reference sample.

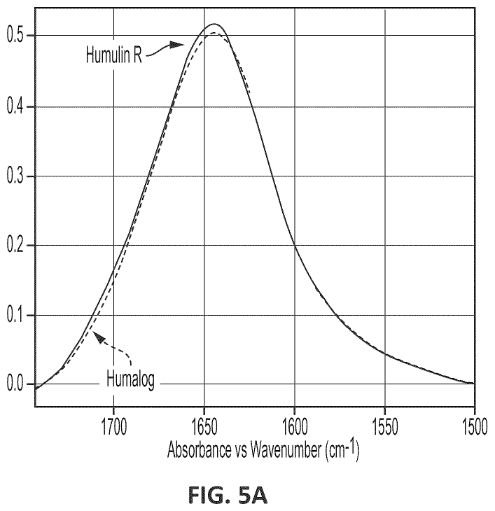

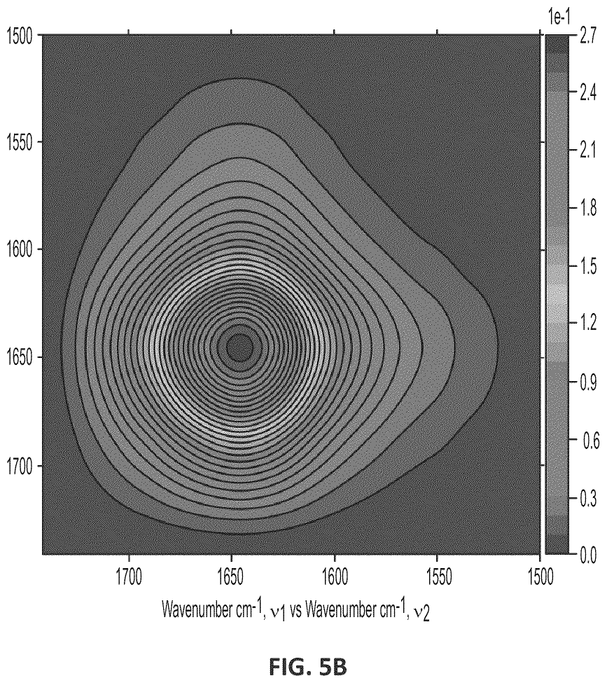

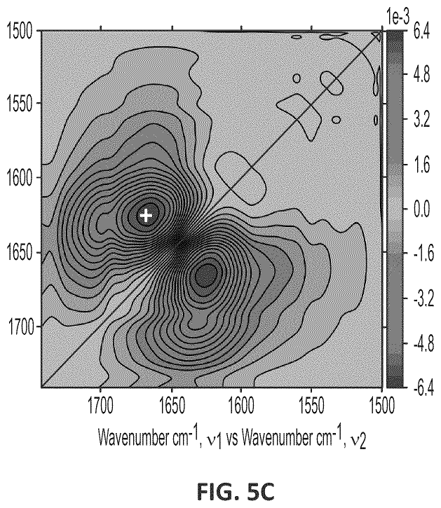

The results of the system and method can also confirm a non-identification of a sample when compared to a reference standard. That is, where the sample being analyzed is not the same as the reference standard, then the 2T2D comparative analysis will indicate this. at least in the asynchronous plot and weighted difference spectra. FIGS. 5A-5C provide an example of how the system and method indicate a non-identification of a sample as a reference standard when the sample differs from the reference. In FIGS. 5A-5C, a comparative analysis was performed comparing a sample of Humulin R against a sample of Humalog, with the spectral evaluation being performed within the spectral region of 1740-1500 cm⁻¹. Although the amino acid sequences for Humulin R and Humalog are identical within the chain A and B except for the proline and lysine, near the C-terminal end of chain B they are inverted from one another. Thus, where Humalog is the reference sample, the process should not indicate a positive identification of Humalog for a sample being analyzed that is Humulin R. For illustrative purposes, the Humalog sample will be discussed as the reference standard, with the Humulin R sample being discussed as the sample whose identity is being assessed. However, the system and method would work in the same way if Humulin R was the reference standard and the Humalog sample was the sample whose identity was being assessed. FIG. 5A illustrates a QCLM spectral overlay generated from acquired spectral images of the Humulin R sample and Humalog reference standard sample. FIG. 5B illustrates the 2T2D synchronous plot generated from spectra for the Humulin R sample and Humalog reference standard sample obtained from the acquired spectral images. FIG. 5C illustrates the 2T2D asynchronous plot generated from spectra for the Humulin R sample and Humalog reference standard sample obtained from the acquired spectral images. The resulting asynchronous plot shown in FIG. 5C illustrates evidence that the Humulin R sample is not the same as the Humalog reference standard sample. That is, the identity of the Humulin R sample is confirmed as not being Humalog. This can be determined due to the presence of cross peaks in the asynchronous plot, which indicate that the sample is not the reference standard. These cross peaks provide information on the key differences between the reference standard sample and the sample being evaluated.

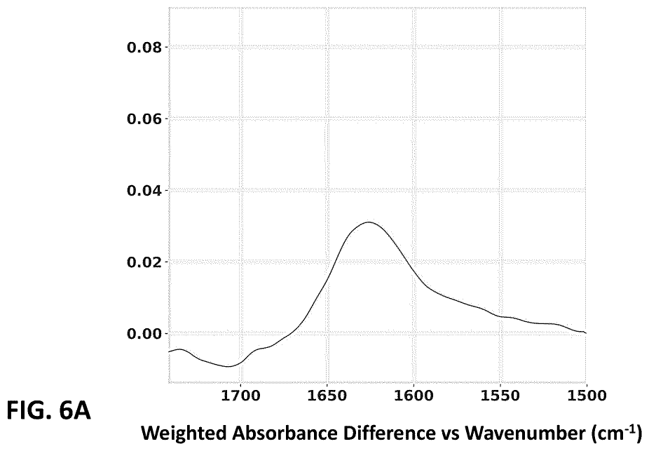

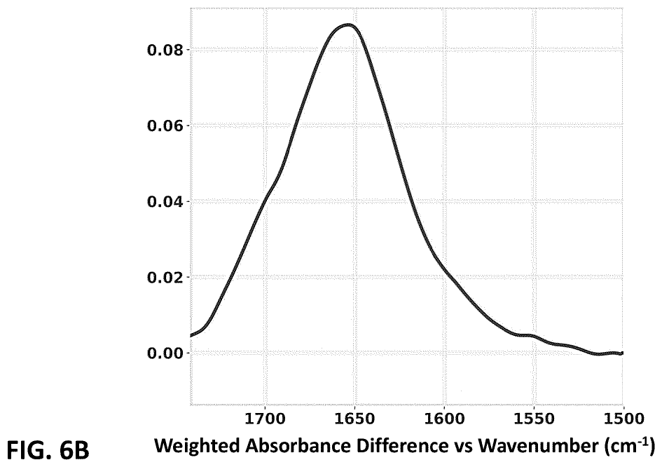

That the Humulin R sample is not Humalog can further be confirmed by assessing weighted difference spectra generated from the 2T2D analysis of the Humulin R sample and Humalog reference sample at room temperature. FIG. 6A illustrates the generated weighted difference spectra for Humulin R, and FIG. 6B illustrates the weighted different spectra for the Humalog reference sample. As shown, the non-identity of the Humulin R sample as being the same as the Humalog reference standard sample is indicated by the weighted absorbance spectral differences between them in FIGS. 6A and 6B. If the weighted difference is within a predetermined threshold, a positive identification is confirmed. Again, for a positive identification, the slope of the weighted difference spectra line would be zero or near zero. Here, the lines in FIGS. 6A and 6B are observed to have varying slopes and changes in absorbance over the spectral range, and thus show that the sample cannot be confirmed to be Humalog (the reference standard).

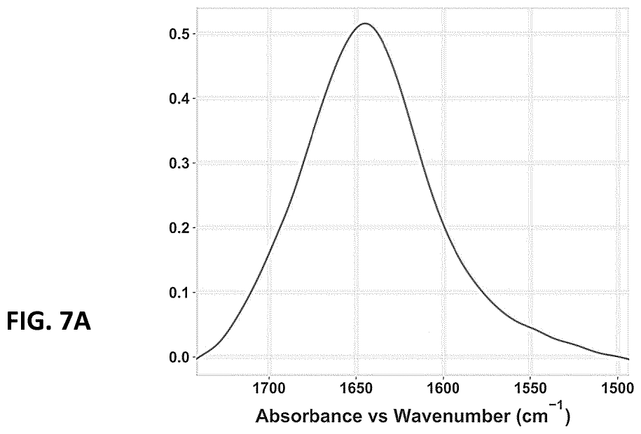

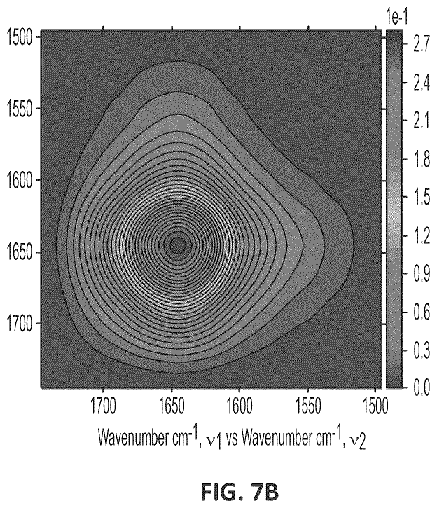

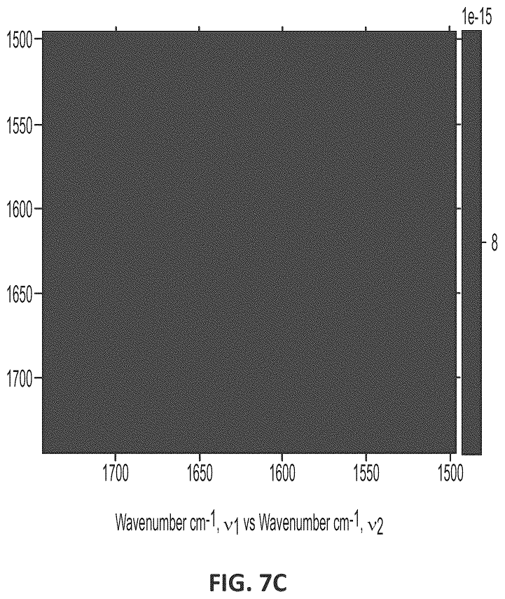

FIGS. 7A-7C provide another illustration of the system and method providing a positive identity confirmation, where the sample being analyzed is the same as the reference sample. In particular, these figures provide an illustration of a Humalog protein identification spectral evaluation within the spectral region of 1740-1500 cm⁻¹ using the protein identity and purity assessment process. This protein identification evaluation includes generating a QCLM spectral overlay of spectral data obtained for two samples of Humalog at room temperature-one of which can be considered a protein sample and the other a known reference sample of Humalog. FIG. 7A illustrates the spectral overlay of the protein sample and reference sample of Humalog at room temperature, with the overlay showing absorbance vs wavenumber. As described above, a 2T2D correlation algorithm is applied, which compares the spectra of the sample being evaluated to the spectra of the reference standard sample. The 2T2D correlation process may be applied to the acquired spectral data to generate a 2T2D synchronous spectrum and a 2T2D asynchronous spectrum. Two contour plots may then be generated from the synchronous and asynchronous spectra—a synchronous plot and an asynchronous plot. FIG. 7B illustrates an example of a 2T2D synchronous plot generated from the spectra data of the sample and reference standard. FIG. 7C illustrates an example of a 2T2D asynchronous plot generated from the spectra data of the sample and reference standard. The resulting 2T2D asynchronous plot shown in FIG. 7C, with no contours or peaks resulting from differences between the sample and reference standard spectra, is a typical result where the sample is identical to the reference standard. Where the sample is not the same as the reference standard, for example where the sample is a different protein than the reference standard protein, then the asynchronous plot would include contours and peaks indicative of the two not being the same.

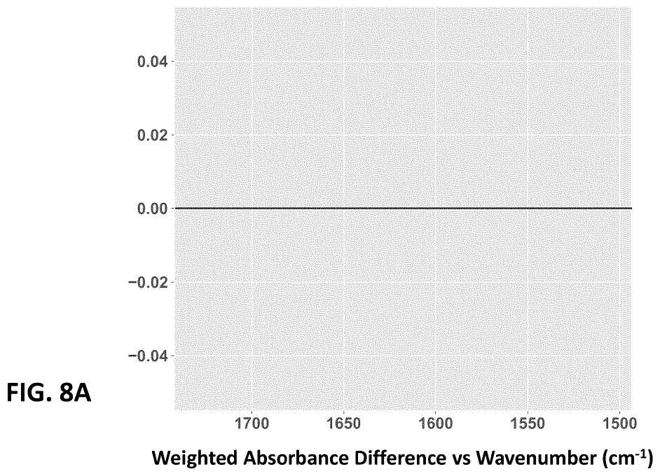

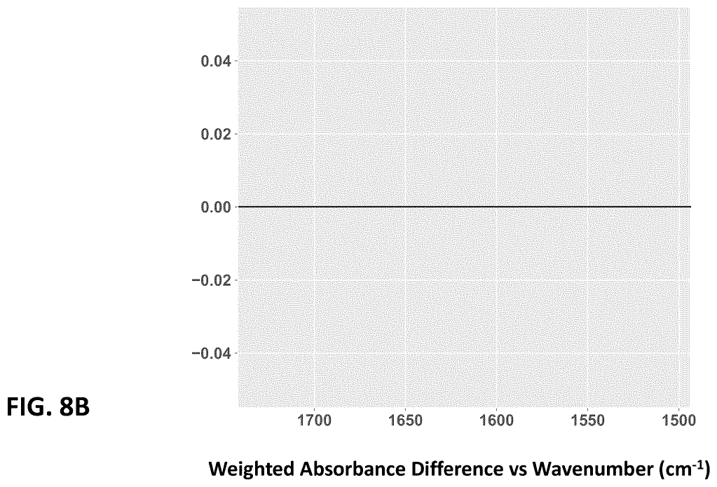

This positive identification can also be confirmed by evaluating the 2T2D weighted difference spectra for the sample and the reference standard sample. FIG. 8A illustrates the 2T2D weighted difference spectra for the Humalog reference standard sample, and FIG. 8B illustrates the 2T2D weighted difference spectra for the sample being analyzed. The weighted difference spectra were generated from data acquired for both the reference standard sample and sample being analyzed at room temperature. As shown, both FIG. 8A and FIG. 8B show a single line with a slope of zero or near zero value. This indicates that there is no difference between the sample being analyzed and the Humalog reference standard sample, confirming the identity of the sample being analyzed as Humalog. That is, when the weighted difference spectra shows only a difference between the sample being analyzed and the reference standard sample that is zero or near zero, then the sample being analyzed can be identified as being the same protein as the reference standard sample. As discussed above with respect to FIGS. 4A-4B, where the weighted difference spectra are within a predetermined threshold or confidence limit, then a positive identification of the sample being the same as the reference standard can be made.

The system and method further may include performing 2D-COS analysis on the spectral images acquired for the sample and the reference standard sample. 2D-COS synchronous and asynchronous plots may be generated, as described above, and peaks in the plots evaluated to determine the specific characteristics, or dynamic fingerprint, of the samples being analyzed. This method also provides enhanced spectral resolution which is key to the comparability assessment between the reference and sample. In particular, the number and position of the cross peaks in the synchronous and asynchronous plots may be used to assess the purity of the protein sample and any differences between the sample and the reference standard.

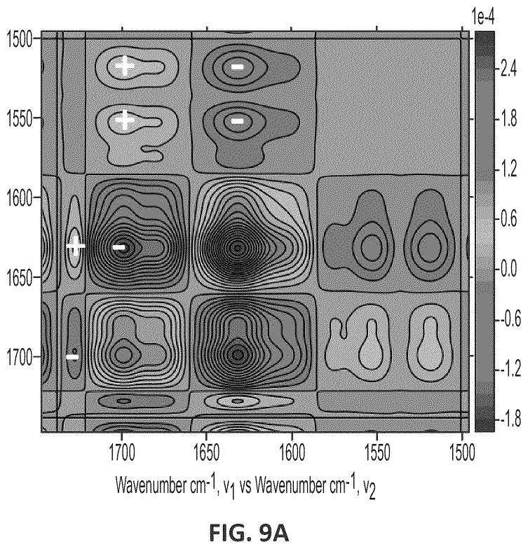

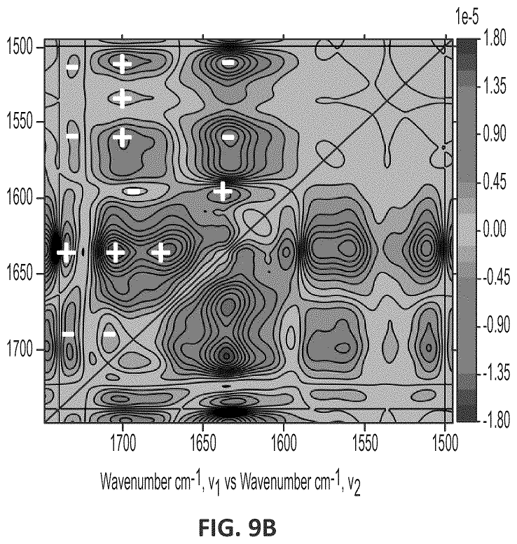

FIG. 9A shows a 2D-COS synchronous plot for a Humulin R sample within the spectral region of 1740-1500 cm⁻¹, generated from spectral data acquired for the temperature range of 26-42° C. with the spectral data acquired at temperature intervals of 4° C. FIG. 9B shows a 2D-COS asynchronous plot for a Humulin R sample within the spectral region of 1740-1500 cm⁻¹, generated from spectral data acquired for the temperature range of 26-42° C. with the spectral data acquired at temperature intervals of 4° C. The cross peaks in FIGS. 9A and 9B are indicated by the “+” (plus) and “−” (minus) signs in the plots. These cross peaks provide the basis for the determination of the dynamic fingerprint associated with the therapeutic protein under the defined stress and formulation conditions. For example, the cross peaks are the determinants of the molecular dynamics of the therapeutic protein in solution during thermal perturbation, which here was an increase in temperature in 4° C. intervals. These cross peaks represent the dynamic fingerprint of the protein. These information rich plots demonstrate the enhanced resolution provided when compared to the overlay of FIG. 2A.

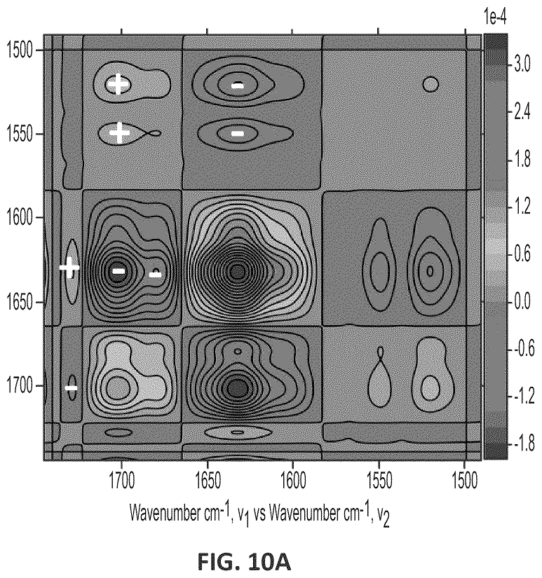

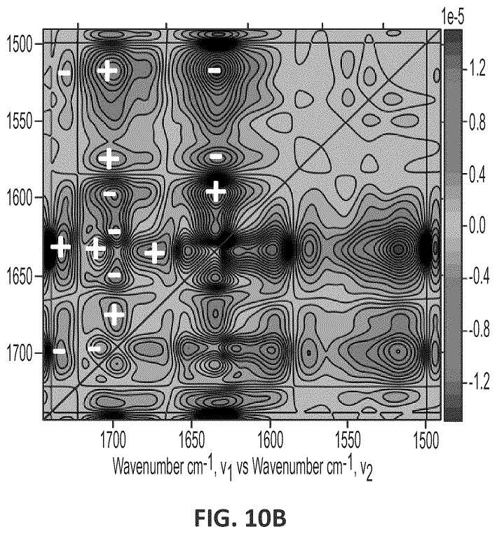

FIG. 10A shows a 2D-COS synchronous plot for a Humalog sample within the spectral region of 1740-1500 cm⁻¹, generated from spectral data acquired for the temperature range of 26-42° C. with the spectral data acquired at temperature intervals of 4° C. FIG. 10B shows a 2D-COS asynchronous plot for a Humulin R sample within the spectral region of 1740-1500 cm⁻¹, generated from spectral data acquired for the temperature range of 26-42° C. with the spectral data acquired at temperature intervals of 4° C. The cross peaks in FIGS. 10A and 10B are indicated by the “+” (plus) and “−” (minus) signs in the plots. These cross peaks provide the basis for the determination of the dynamic fingerprint associated with the therapeutic protein under the defined stress and formulation conditions. For example, the cross peaks are the determinants of the molecular dynamics of the therapeutic protein in solution during thermal perturbation, which here was an increase in temperature in 4° C. intervals. These cross peaks represent the dynamic fingerprint of the protein. These information rich plots demonstrate the enhanced resolution provided when compared to the overlay of FIG. 7A.

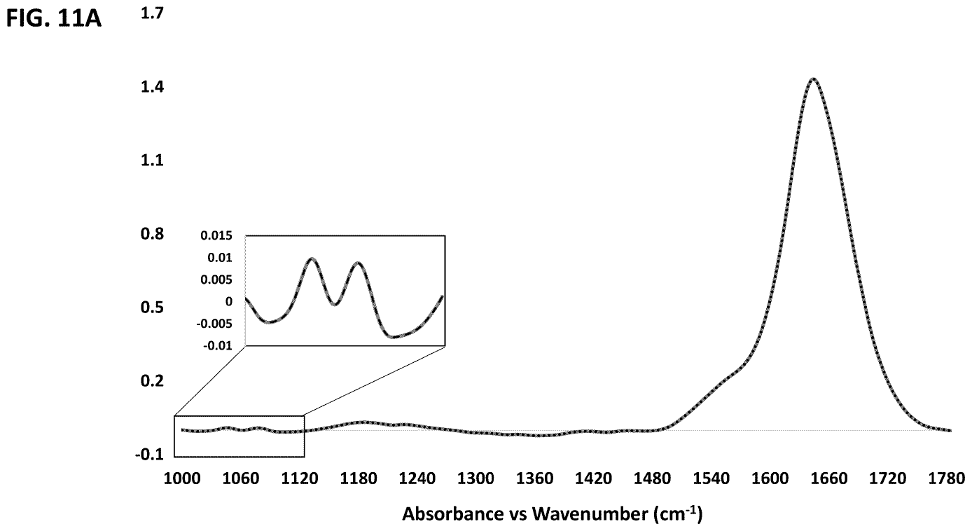

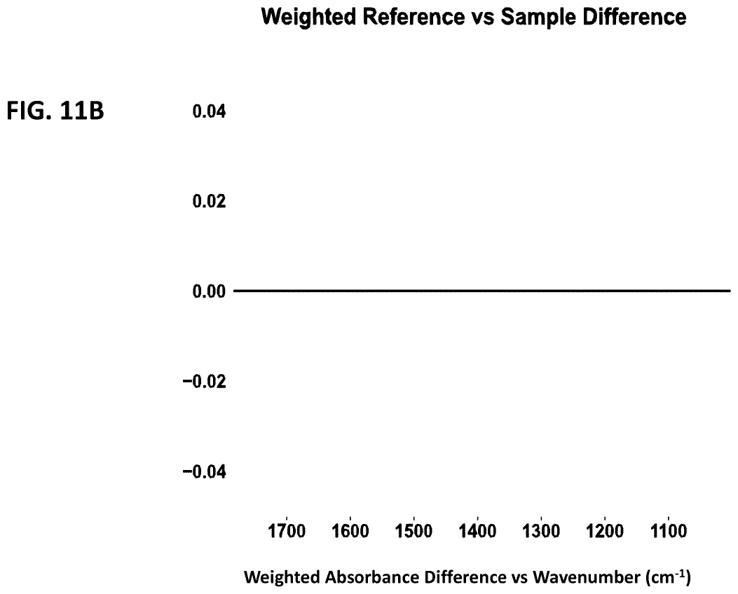

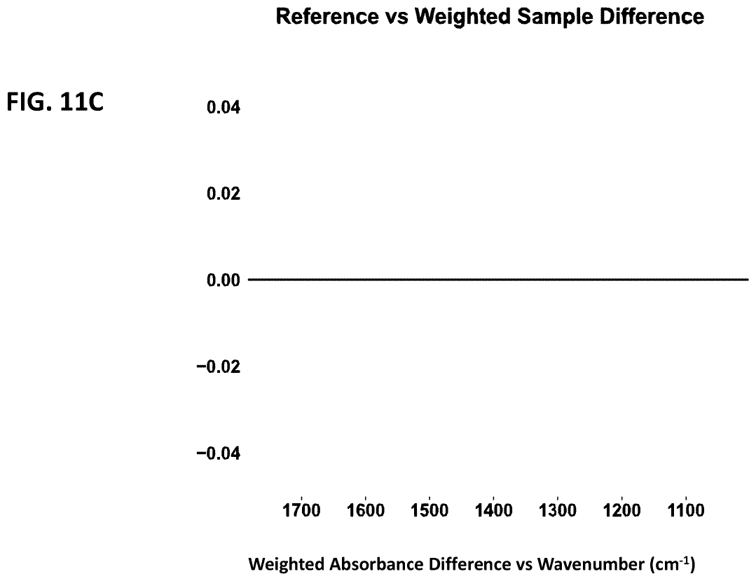

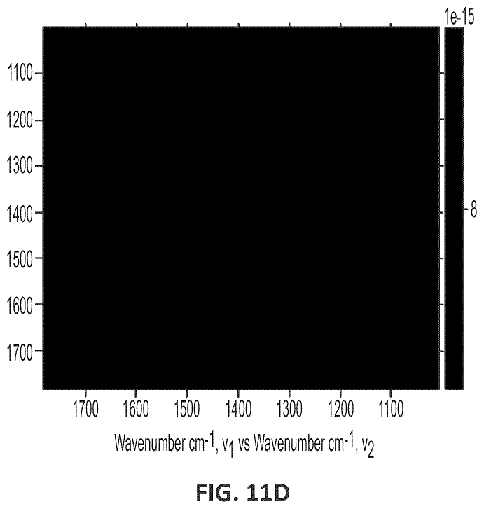

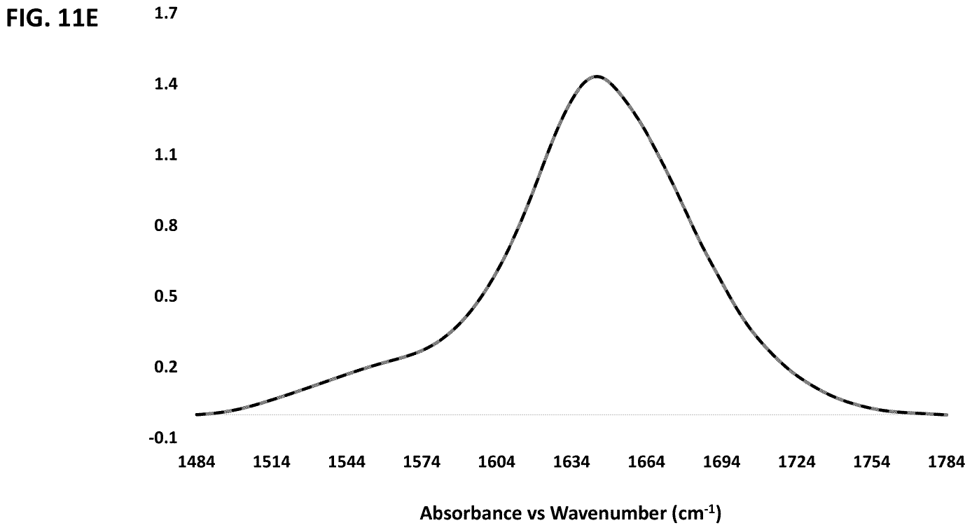

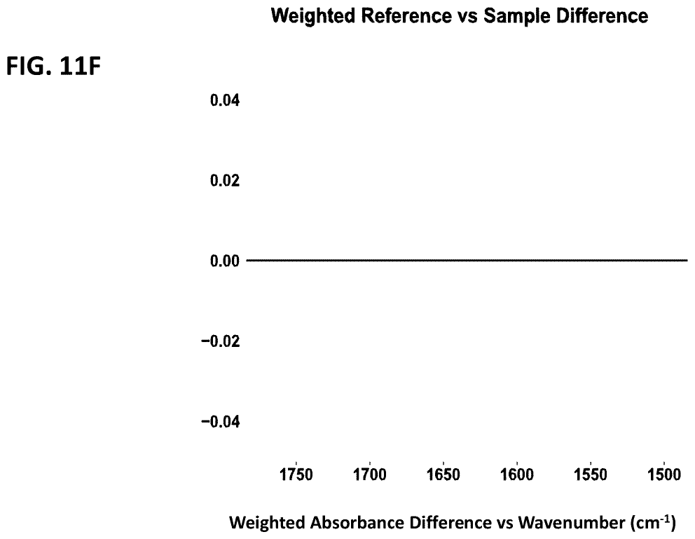

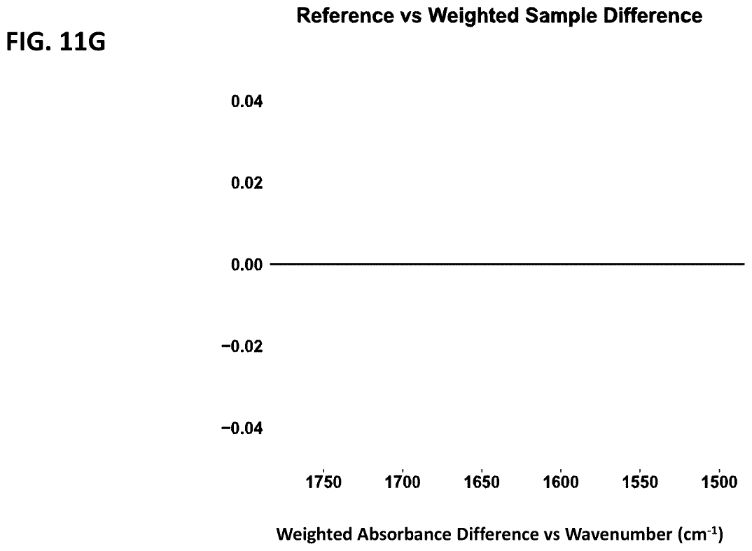

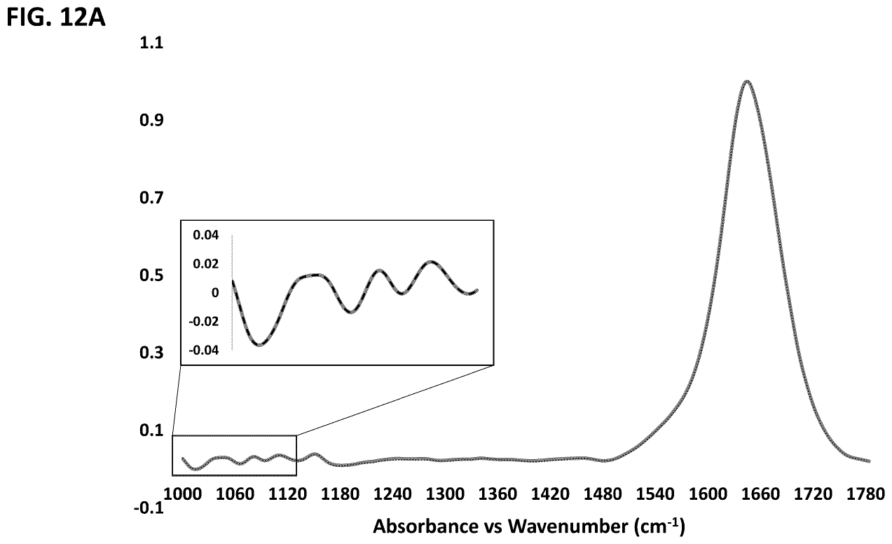

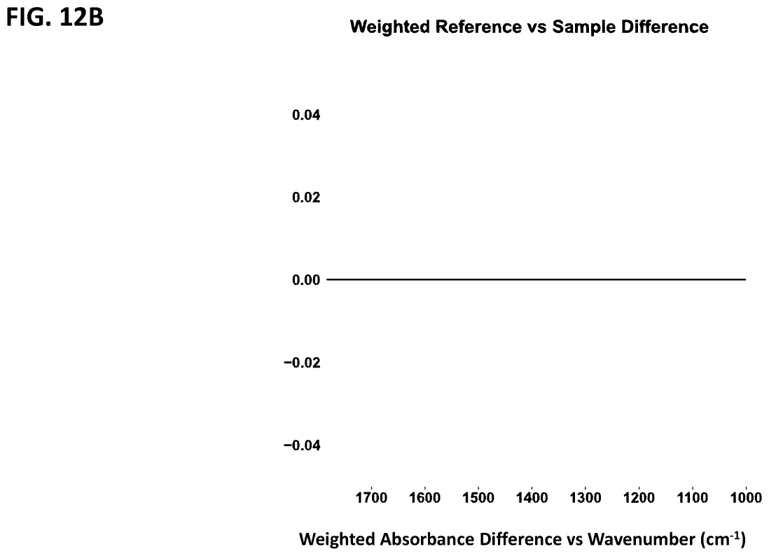

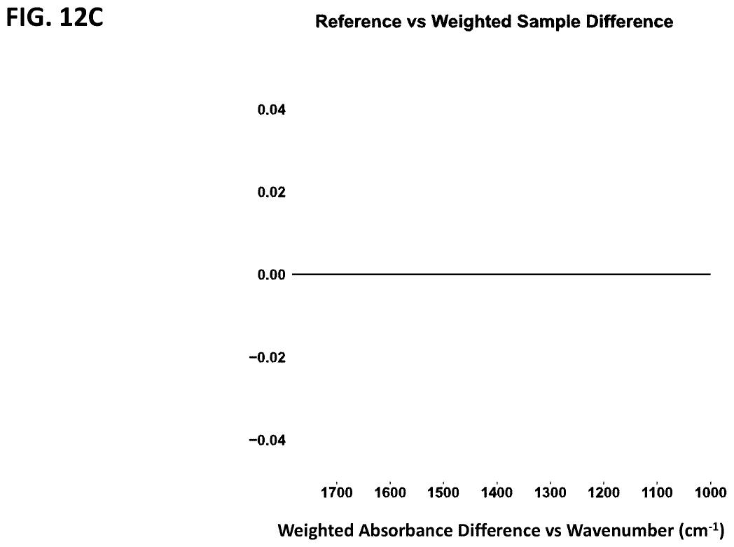

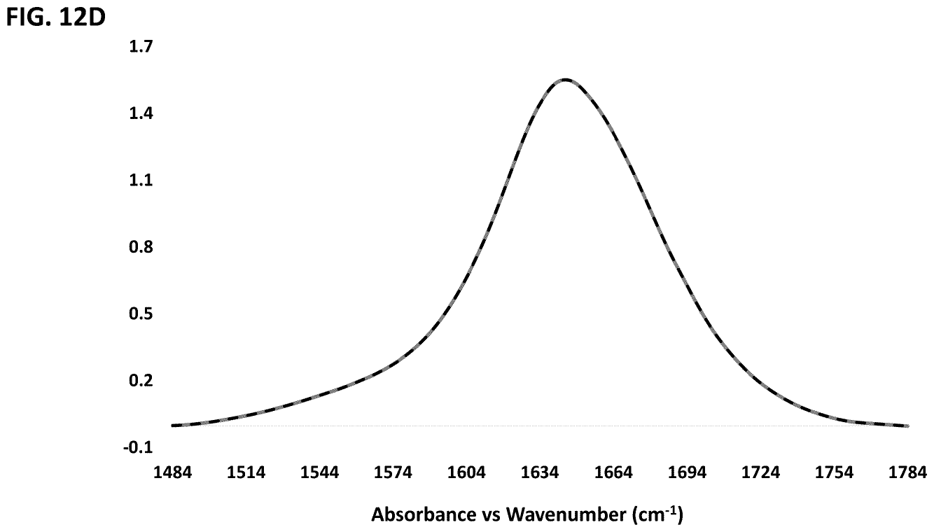

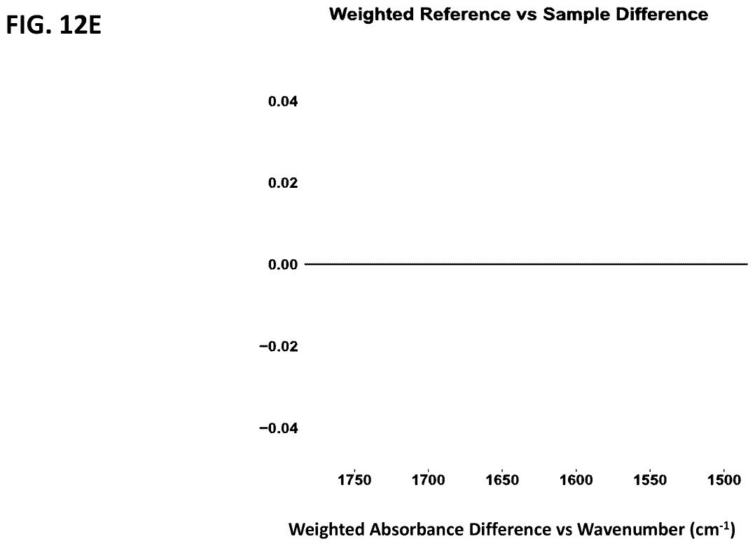

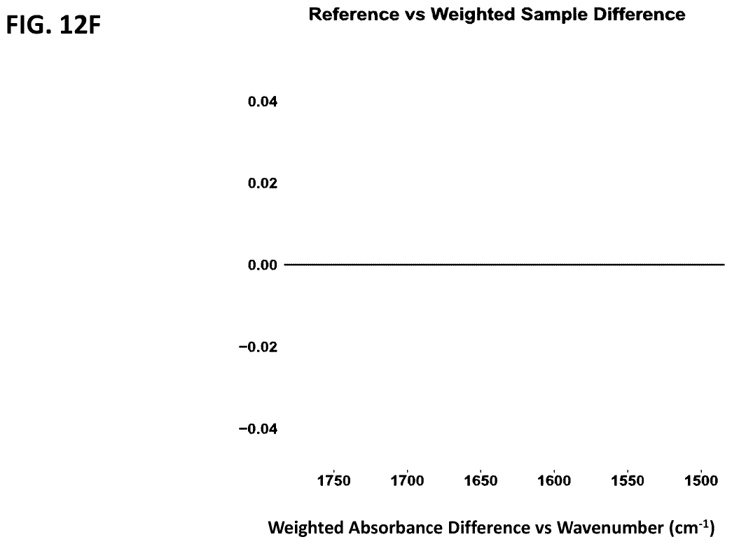

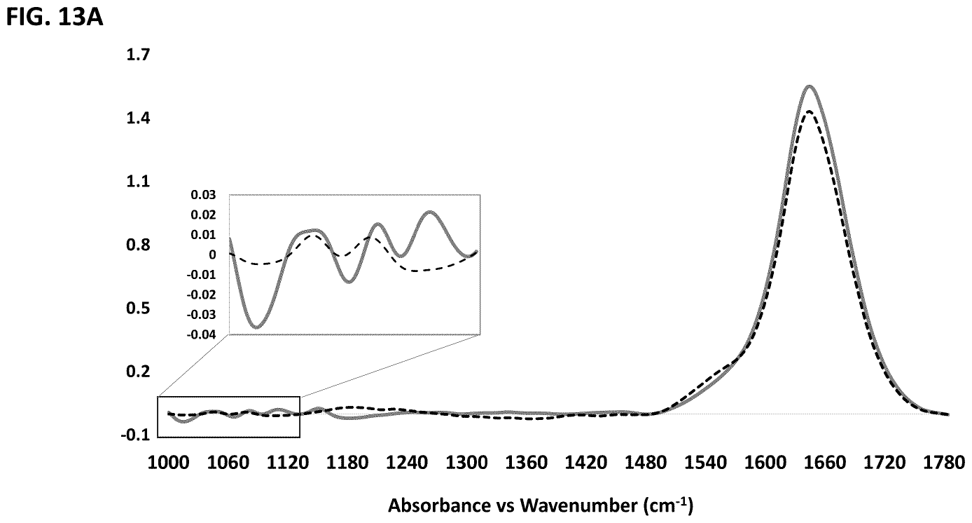

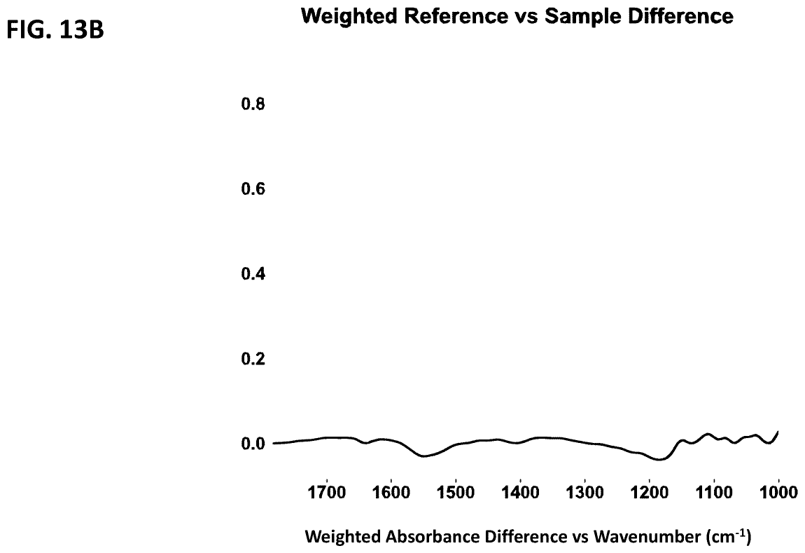

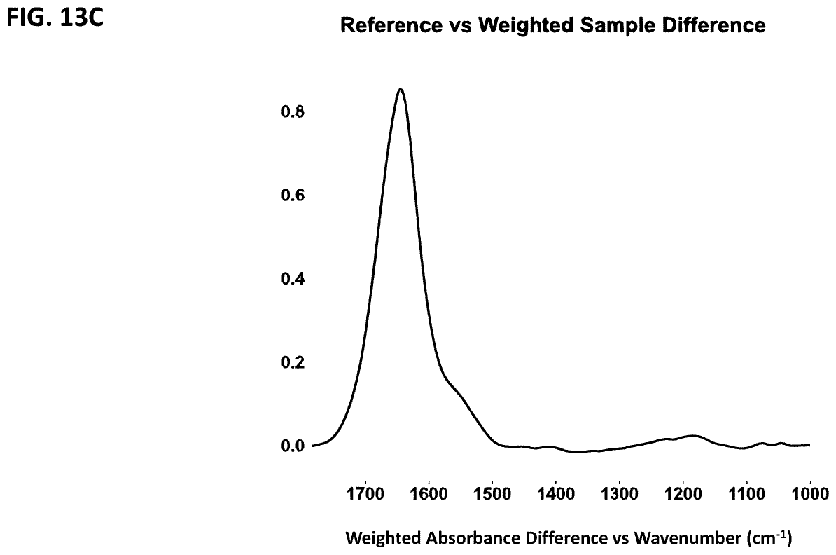

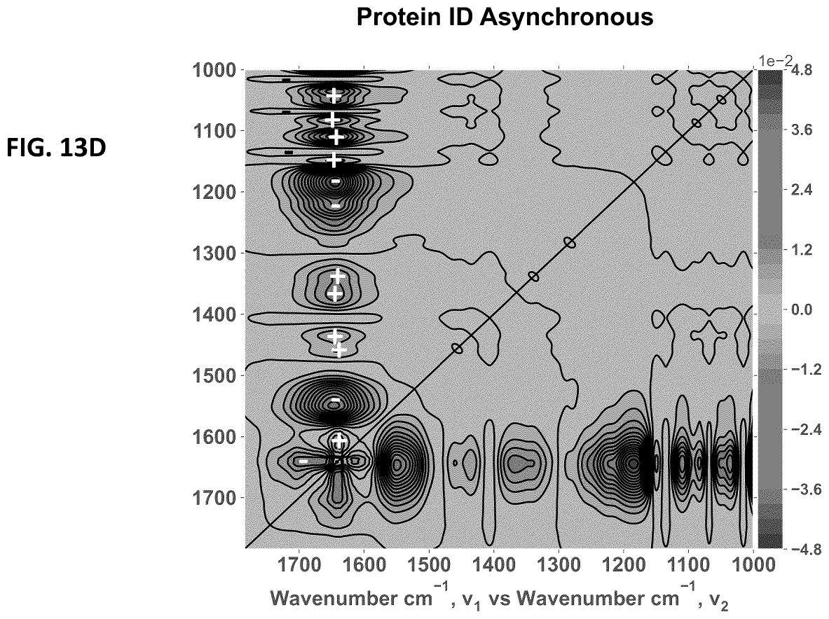

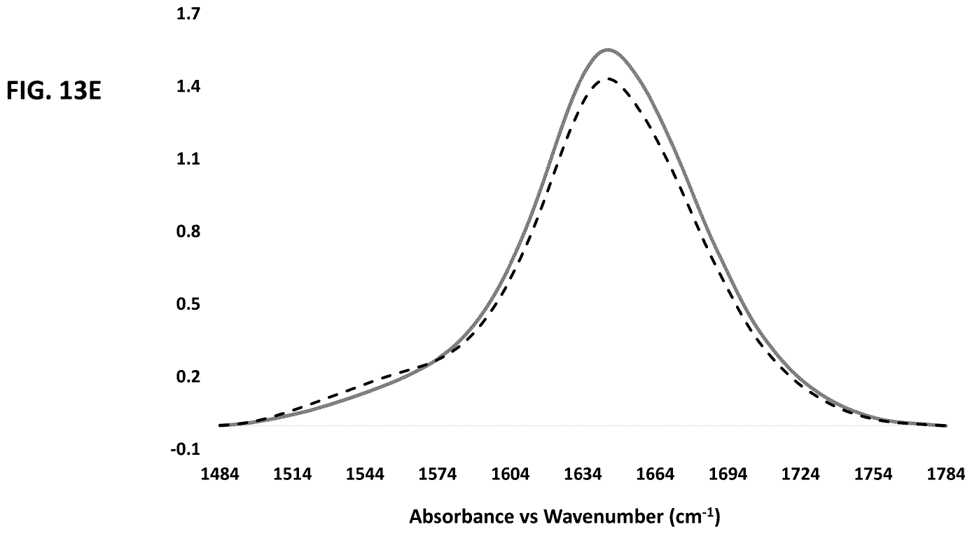

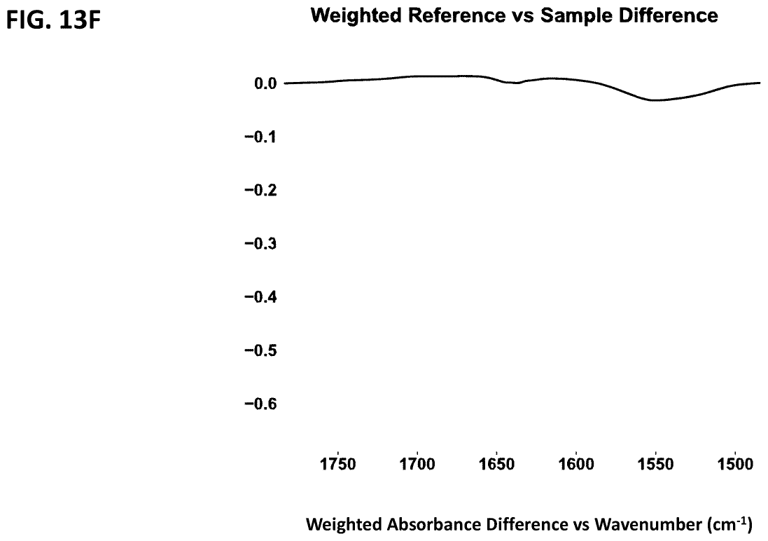

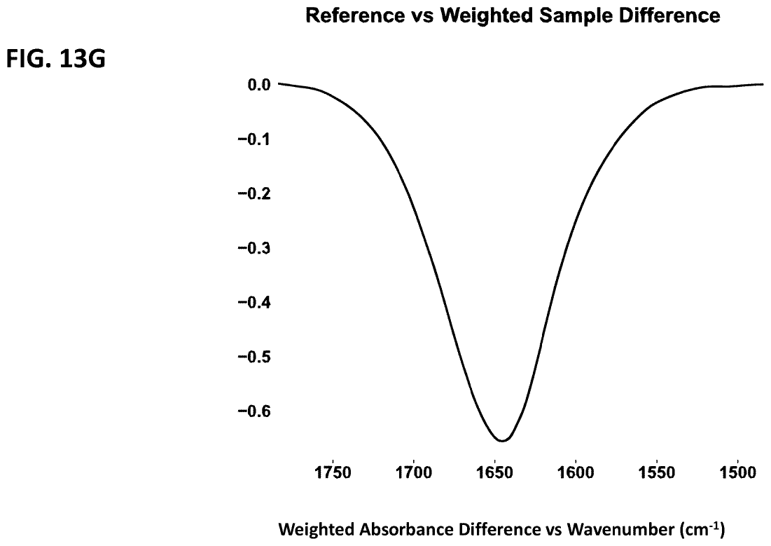

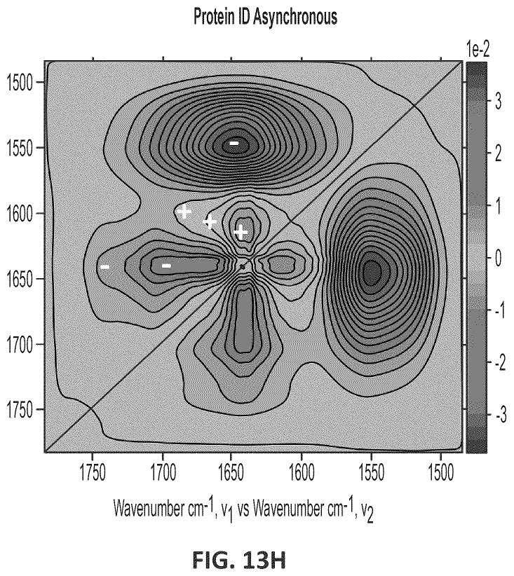

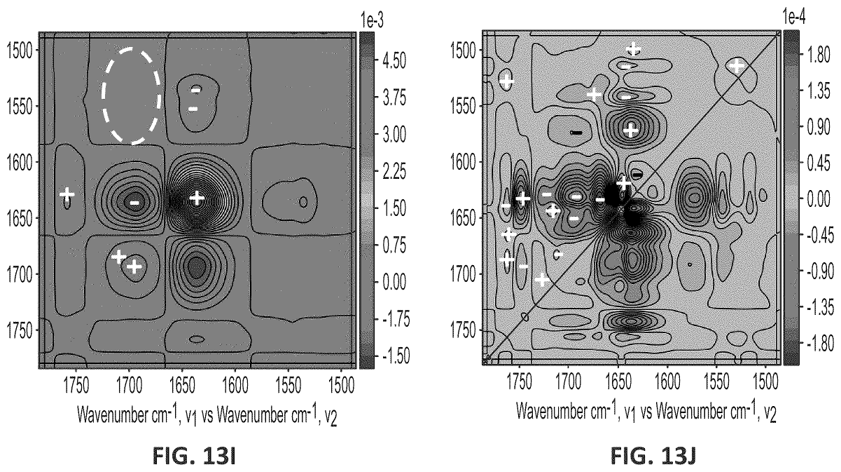

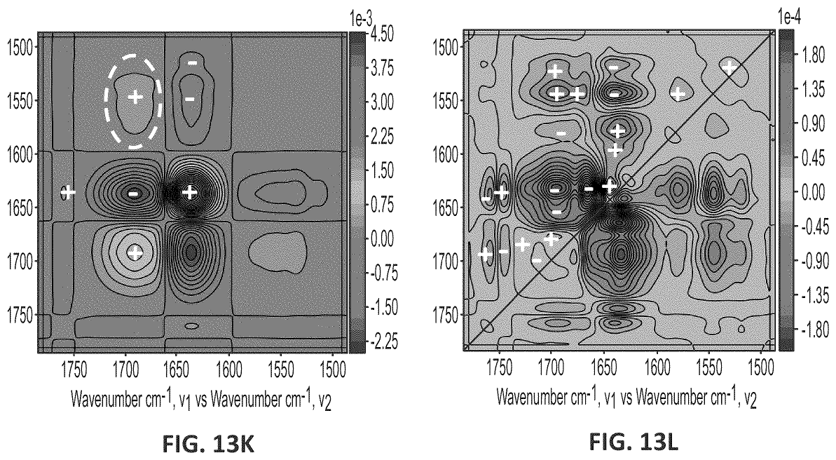

The method described herein can evaluate multiple types of proteins. As shown in FIGS. 11A-12F, for example, the method canevaluate samples of a protein known to be the same as a reference protein to establish spectral identity. And, as shown in FIGS. 13A-15H, the method can further be used to evaluated samples to establish differences or non-identity between a sample and a reference. The quantitative results of such evaluations are summarized in Table I, included later herein.

The method described herein can be employed to ascertain confirmation of protein ID of numerous different types of samples including, for example, commercial peptides such as insulin, engineered monoclonal antibodies, and glycosylated extracellular domain targets. For example, in evaluating such samples, the method demonstrated usefulness in assessment of purity for the glycosylated target when evaluated against a mixture of the target and a reference monoclonal antibody.

FIG. 11A provides an illustration of QCLM spectral overlays for a sample comprised of an engineered monoclonal antibody 1 (mAb1) and its reference at 25° C. within two spectral regions of interest: (full region) 1780-1015 cm⁻¹ and, as shown in the blow up in the insert, the mAb1 glycan spectral region selected within the spectral region of 1134-1015 cm⁻¹ as a critical component of the evaluation to establish identity based on the post-translational modification of glycosylation to ensure the identity of the engineered monoclonal antibody. FIG. 11B is an illustration of a 2T2D weighted difference spectra for the reference sample of mAb1 within the full spectral region of 1780-1015 cm⁻¹. FIG. 11C is an illustration of a 2T2D weighted difference spectra for the sample of mAb1 within the full spectral region of 1780-1015 cm⁻¹. FIG. 11D is an illustration of the 2T2D asynchronous plot, which is featureless due to the identity of the mAb1 sample matching that of the mAb1 reference, and thus there are no differences between the two which leads to no contours or peaks in the asynchronous plot. This confirms the identity and purity of the mAb1 sample in the evaluation. The sensitivity of the 2T2D asynchronous plot is demonstrated with its bar scale on the right hand side at 1×10⁻¹⁵ A.U. FIG. 11E shows an illustration of a spectral overlay of an mAb1 and a reference sample of mAb1 at room temperature within the spectral region 1780-1486 cm⁻¹. The 2T2D analysis of mAb1 within the amide I and II band region allow for the evaluation of the presence of aggregates and degradation products in the sample as well as the structural similarity of the mAb1 sample when compared to its reference sample. FIG. 11F is an illustration of a 2T2D weighted difference spectra for the reference sample of mAb1 within the amide I and II band spectral region of 1780-1486 cm⁻¹. FIG. 11G is an illustration of a 2T2D weighted difference spectra for the sample of mAb1 within the spectral region of 1780-1486 cm⁻¹.

Similar results were obtained for mAb2, a second engineered monoclonal antibody 2 (mAb2), and its reference at 25° C. within two spectral regions of interest: (full region) 1780-1015 cm⁻¹ and the (amide I and II bands) 1780-1486 cm⁻¹ as shown in Table I below. The 2T2D analysis involved the generation of the weighted difference spectra and asynchronous plot for each spectral region, as is summarized in Table 1. The method allows for the comprehensive assessment of mAb of interest which also includes the evaluation of post-translational modifications within the same assay. Thus, the method provides an understanding of the protein candidate attributes in minutes.

FIG. 12A shows an illustration of the spectral overlay of a glycosylated extracellular domain target sample and a reference sample of the glycosylated target at room temperature within the spectral region of 1780-1015 cm⁻¹ based on spectral data acquired using a QCLM. FIG. 12A includes a blow up insert showing the target glycan spectral region selected within the spectral region of 1134-1015 cm⁻¹. The benefits of the method to include in its analysis the full spectral region is demonstrated by recognizing the same glycosylation associated vibrational modes for the target as a direct evaluation of the glycosylation pattern regarding the carbohydrate composition of the protein's glycan composition. FIG. 12B is an illustration of a 2T2D weighted difference spectra for the reference sample of a glycosylated target within the spectral region of 1780-1015 cm⁻¹). The weighted difference spectra numerical analysis is further illustrated in Table 1 below. FIG. 12C is an illustration of a 2T2D weighted difference spectra for the sample of a glycosylated target within the spectral region of 1780-1015 cm⁻¹. FIG. 12D shows an illustration of the spectral overlay of a glycosylated extracellular domain target and a reference sample of the glycosylated target at room temperature within the amide I and II band spectral region 1780-1485 cm⁻¹ acquired using a QCLM. FIG. 12E shows a resulting weighted difference spectrum from the 2T2D analysis resulting from the spectral comparison of two glycosylated extracellular target samples within the amide I and II band region of 1780-1486 cm⁻¹. FIG. 12F is an illustration of a 2T2D weighted difference spectra for the sample of a glycosylated target within the spectral region of 1780-1485 cm⁻¹.

FIG. 13A shows an illustration of the spectral overlay of an mAb1 sample and a reference sample of a glycosylated target at room temperature within the spectral region 1780-1015 cm⁻¹ based on spectral data acquired using a QCLM. Differences in the selected glycan region are shown in the spectral region of 1134-1015 cm⁻¹, as shown in the zoomed in blowout in FIG. 13A, demonstrate the powerful approach of evaluating this spectral region due its selectivity and sensitivity to the vibrational modes associated with the glycan structure.

FIG. 13B is an illustration of a 2T2D weighted difference spectra for the reference sample of a glycosylated target within the spectral region of 1780-1015 cm⁻¹. FIG. 13C is an illustration of a 2T2D weighted difference spectra for the sample of mAb1 within the full spectral region of 1780-1015 cm⁻¹. The weighted spectral similarity was assessed using a Euclidian distance algorithm as described above, and the Euclidian distance value was determined to be 6.52, therefore non identity to the reference sample was determined. Such results are shown in Table 1. FIG. 13D is an illustration of the 2T2D asynchronous plot of the mAb1 sample when compared to the glycosylated target reference sample within the full spectral region of 1780-1015 cm⁻¹. The cross peak assignments for the 2T2D asynchronous plot within the full spectral region of 1780-1015 cm⁻¹ are summarized in Tables II-A and II-B, provided below. FIG. 13E shows an illustration of the spectral overlay of an mAb1 sample and a reference sample of the glycosylated target at room temperature within the spectral region 1780-1486 cm⁻¹ based on spectral data acquired using a QCLM. Differences in intensity contributions can be observed for both the amide I and II bands providing the basis for the spectral comparison within the amide I and II band spectral region of 1780-1486 cm⁻¹. These intensity differences within the amide 1 band (1700-1600 cm⁻¹) are structurally related, while changes in intensity within both the amide I and II bands are related to both amino acid composition and their weak interactions. FIG. 13F is an illustration of a 2T2D weighted difference spectra for the reference sample of a glycosylated target within the spectral region of 1780-1486 cm⁻¹. FIG. 13G is an illustration of a 2T2D weighted difference spectra for the sample of mAb1 within the amide I and II band spectral region of 1780-1486 cm⁻¹. FIG. 13H is an illustration of the 2T2D asynchronous plot of the mAb1 sample when compared to the glycosylated target reference sample within the spectral region of 1780-1486 cm⁻¹. The cross peak assignment for the 2T2D asynchronous plot within the amide I and II band spectral region of 1780-1486 cm⁻¹ are summarized in Table II-C and Table II-D below. Also, the 2T2D asynchronous plot provides an increased spectral resolution of the spectral differences observed when comparing these spectra in the form of the cross peaks and their assignments. The 2T2D asynchronous plot further summarizes the different vibrational mode contributions due to the structural and intra-molecular interaction differences observed for these two proteins providing the basis for an unambiguous means for comparative analysis. FIG. 13I shows a 2D-COS synchronous plot for an mAb1 sample within the spectral region of 1780-1486 cm⁻¹, generated from spectral data acquired for the temperature range of 24-52° C. with the spectral data acquired at temperature intervals of 4° C. (as shown in Table II-E below). The mAb1 protein was not observed to aggregate due to thermal perturbation. FIG. 13J shows a 2D-COS asynchronous plot for the mAb1 sample within the spectral region of 1780-1486 cm⁻¹, generated from spectral data acquired for the temperature range of 24-52° C. within the spectral data acquired at temperature intervals of 4° C. (as shown in Table II-F). FIG. 13K shows a 2D-COS synchronous plot for a glycosylated target sample within the spectral region of 1780-1486 cm⁻¹, generated from spectral data acquired for the temperature range of 24-52° C. with the spectral data acquired at temperature intervals of 4° C. (as shown in Table II-G). FIG. 13L shows a 2D-COS asynchronous plot for a glycosylated target sample within the spectral region of 1780-1486 cm⁻¹, generated from spectral data acquired for the temperature range of 24-52° C. with the spectral data acquired at temperature intervals of 4° C. (as shown in Table II-H). This analysis illustrates the differences in the dynamic behavior of these proteins observed during thermal stress, and demonstrates the spectral comparison at different temperatures to confirm identity and purity of a protein sample against its reference standard.

The method described herein was also used to conduct spectral evaluation of an mAb2 sample against a glycosylated target, which like monoclonal antibodies are comprised of mainly β sheet secondary structure. Spectral overlays and associated plots were produced for both the full spectral region of 1780-1015 cm⁻¹ and the amide I and II bands within the 1780-1486 cm⁻¹, with the results being summarized for the 2T2D analysis in Tables III-A, III-B, III-C, and III-D, respectively. The 2T2D asynchronous cross peak assignments are summarized in Tables II-A and II-B, for the spectral region of 1780-1015 cm⁻¹, and provide the detailed differences in spectral contribution observed which also includes different glycosylation vibrational modes. In addition, a separate evaluation was performed for the amide I and II bands. Specifically, the spectral region of 1780-1486 cm⁻¹ region. The differences observed, including by analyzing cross peaks present in 2T2D asynchronous plots, are not limited to structural contributions, but also include differences within side chain vibrational modes and their interactions for the sample when compared to the reference. For example, the asparagine residues located to the β-sheet and the random coil, histidine residue located within the hinge loop and lysine residues located within the β-sheet and random coil in mAb1 that are not present in the reference provide the basis for the molecular differences the non-identity evaluation (summarized in Table II-C). Similarly, for the reference the observed molecular differences include the aspartic acid and glutamic acid residues that are that exhibit variability in hydrogen bonded states due to their exposure to the aqueous environment and the location of glutamates, lysine and aromatic residues (phenylalanine, tyrosine and tryptophan) to the α-helix as summarized in Table II-D. In addition, 2D-COS analysis was performed to establish the molecular dynamics within the temperature range of 24-52° C. for mAb2, as summarized in Tables III-E and III-F for the synchronous and asynchronous plots, respectively. The analysis included the different hydrogen bonded states (as shown in Table III-F) for both aspartic and glutamic acids allowing for the probing of aqueous solvent accessibility of these residues within mAb2. The glycosylated target dynamic fingerprint can be analyzed from the synchronous and asynchronous plots over time. For example, by analyzing changes in the plots over time, the stability or glycan composition differences can be observed.

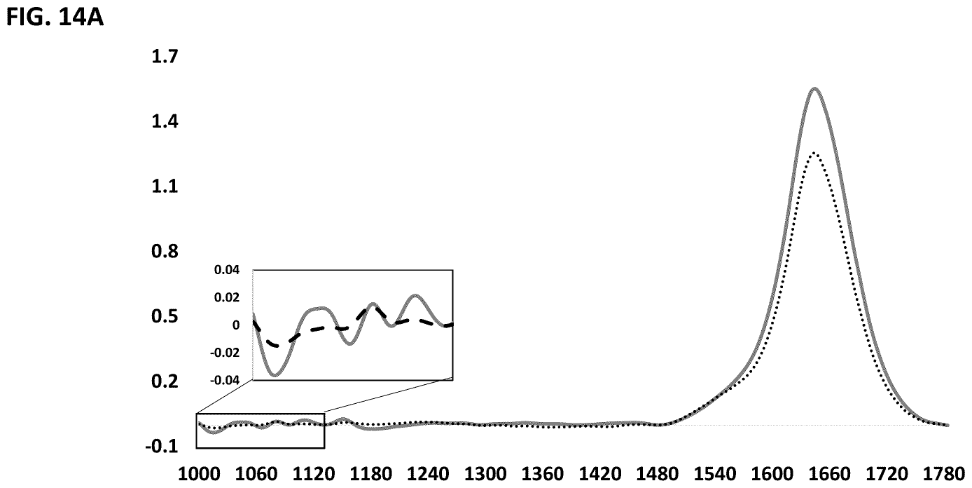

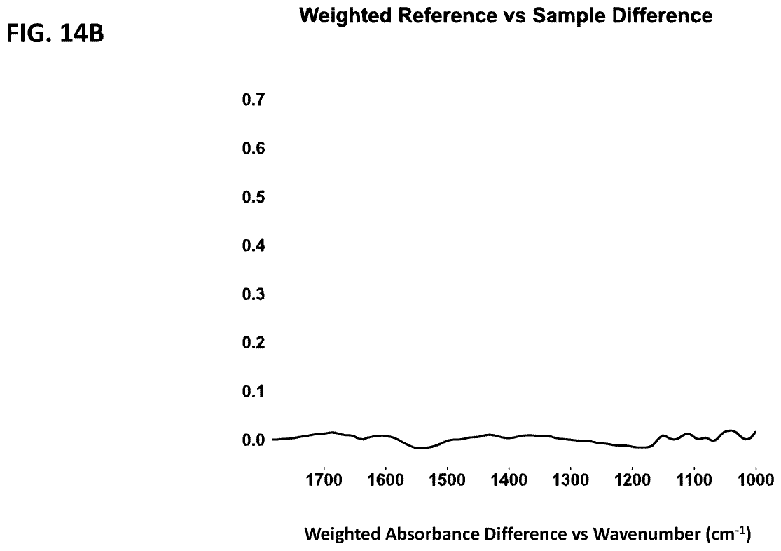

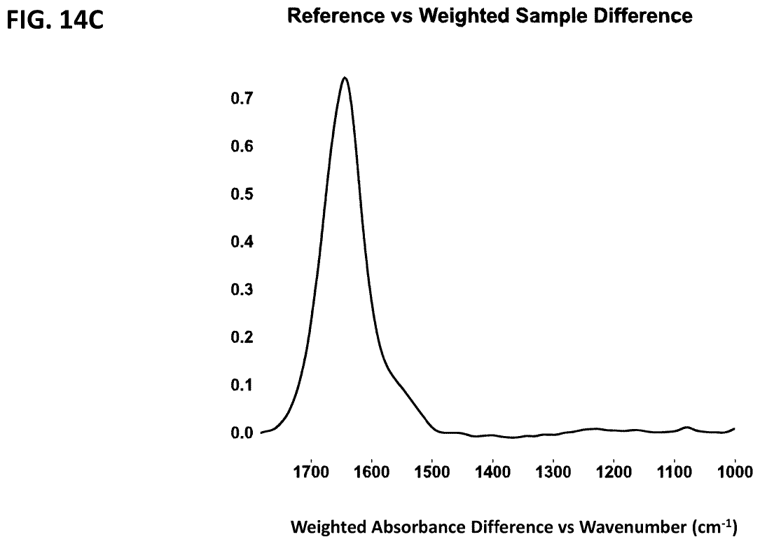

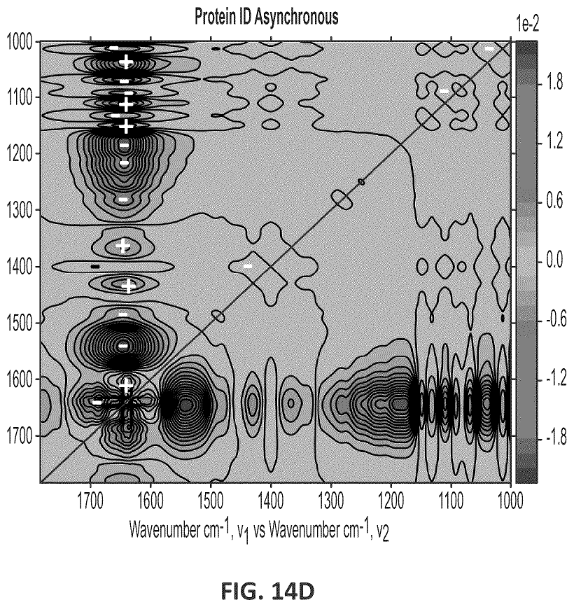

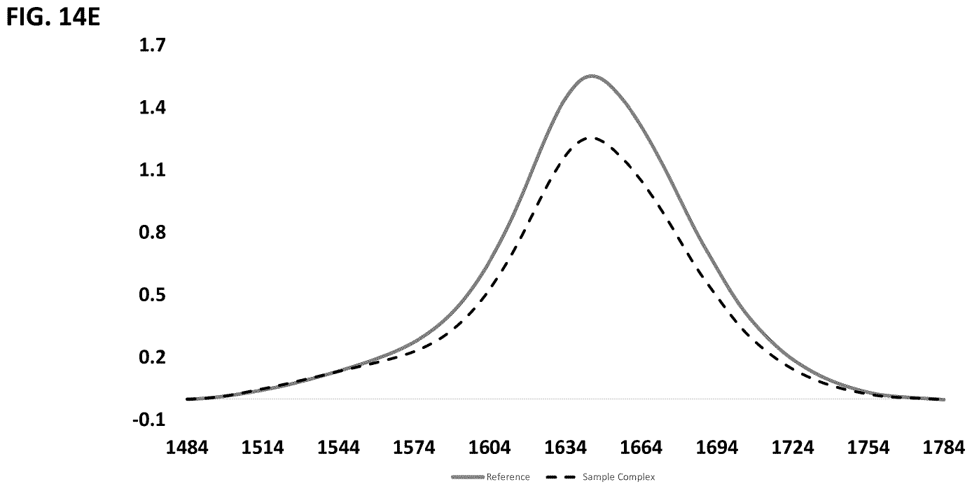

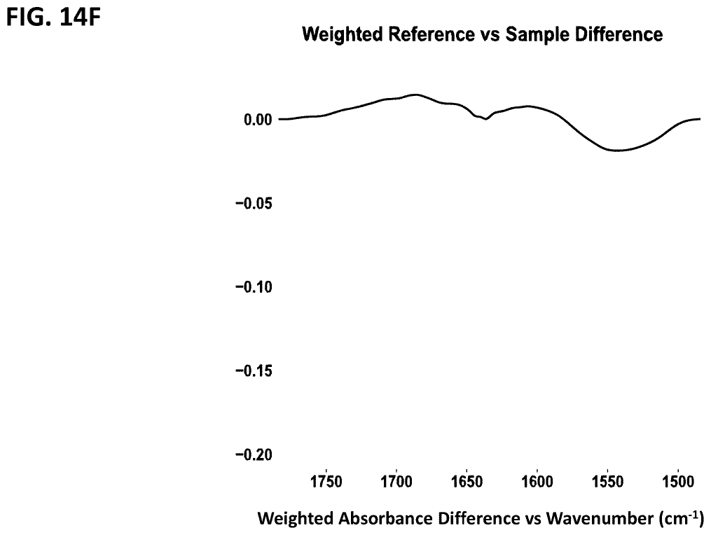

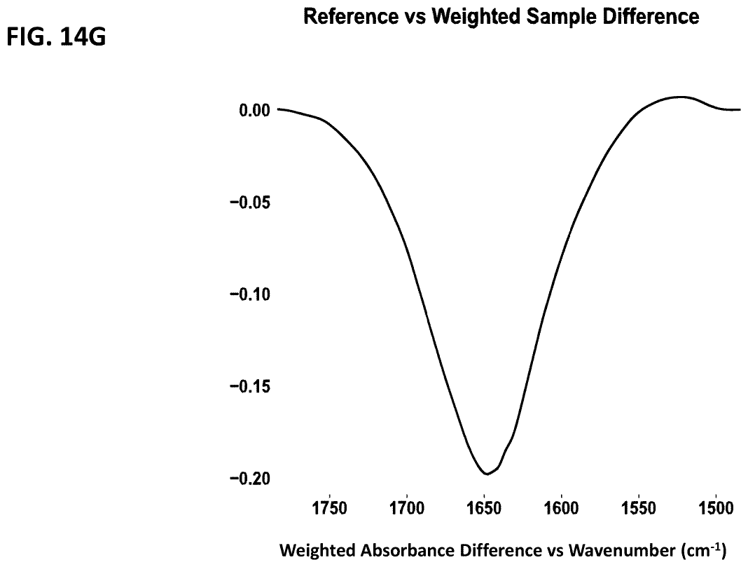

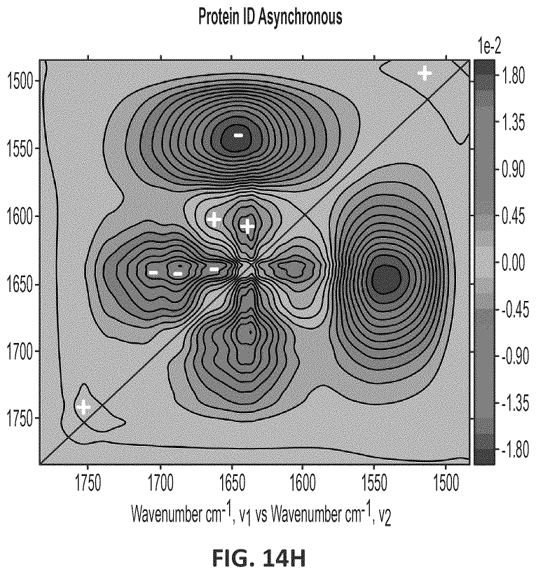

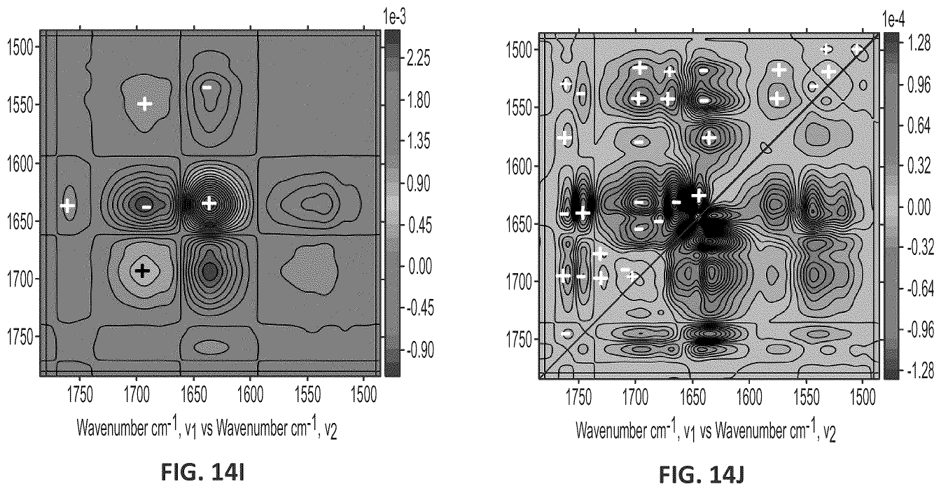

FIG. 14A shows an illustration of the spectral overlay of a glycosylated target/mAb2 (2.0/1.0, mol ratio) mixture sample and a reference sample of the pure glycosylated target at room temperature within the spectral region 1780-1015 cm⁻¹ acquired using a QCLM. Differences in the selected glycan region are shown in the zoomed in insert and demonstrate that even in the case of a mixture the method can evaluate this spectral region due its selectivity and sensitivity to the vibrational modes associated with the glycan structure. FIG. 14B is an illustration of a 2T2D weighted difference spectra for the reference sample of a glycosylated target within the spectral region of 1780-1015 cm⁻¹. FIG. 14C is an illustration of a 2T2D weighted difference spectra for the sample of glycosylated target/mAb2 (2.0/1.0, mol ratio) mixture within the full spectral region of 1780-1015 cm⁻¹. FIG. 14D is an illustration of the 2T2D asynchronous plot of the glycosylated target/mAb2 (2.0/1.0, mol ratio) mixture sample and a reference sample of the pure glycosylated target within the full spectral region of 1780-1015 cm⁻¹. The 2T2D asynchronous plot cross peaks can be evaluated to determine differences in glycan composition/interaction within the glycosylated target when compared to the glycosylated target/mAb2 mixture (as shown in Tables IV-A and IV-B). FIG. 14E shows an illustration of the spectral overlay of a glycosylated target/mAb2 (2.0/1.0, mol ratio) mixture sample and a reference sample of the pure glycosylated target at room temperature within the spectral region 1780-1486 cm⁻¹ acquired using a QCLM. FIG. 14F is an illustration of a 2T2D weighted difference spectra for the reference sample of a glycosylated target within the amide I and II band spectral region of 1780-1486 cm⁻¹. FIG. 14G is an illustration of a 2T2D weighted difference spectra for the sample of glycosylated target/mAb2 (2.0/1.0, mol ratio) mixture within the spectral region of 1780-1486 cm⁻¹. FIG. 14H is an illustration of the 2T2D asynchronous plot of the glycosylated target/mAb2 (2.0/1.0, mol ratio) mixture sample and a reference sample of the pure glycosylated target within the amide I and II band spectral region of 1780-1486 cm⁻¹. In this case, the 2T2D asynchronous plot cross peaks can be evaluated to establish the existence of aspartates and glutamates in the non-hydrogen bonded state (1754, 1746, 1742 cm⁻¹) within the glycosylated target that were no longer exposed to their aqueous environment due to the interaction with mAb2 (as shown in Tables IV-C and IV-D). Once again, the results obtained demonstrate that the method can establish spectral discrimination and determination of non-identity. FIG. 14I shows a 2D-COS synchronous plot for a glycosylated target/mAb2 (2.0/1.0, mol ratio) mixture sample within the spectral region of 1780-1486 cm⁻¹, generated from spectral data acquired for the temperature range of 24-52° C. with the spectral data acquired at temperature intervals of 4° C. FIG. 14J shows a 2D-COS asynchronous plot for a glycosylated target/mAb2 (2.0/1.0, mol ratio) mixture sample within the spectral region of 1780-1486 cm⁻¹, generated from spectral data acquired for the temperature range of 24-52° C. with the spectral data acquired at temperature intervals of 4° C. The cross peak assignments for both the synchronous and asynchronous plots are summarized in Tables IV-E and IV-F, represent the complexity of the dynamics observed for mAb2 interacting with its glycosylated target.

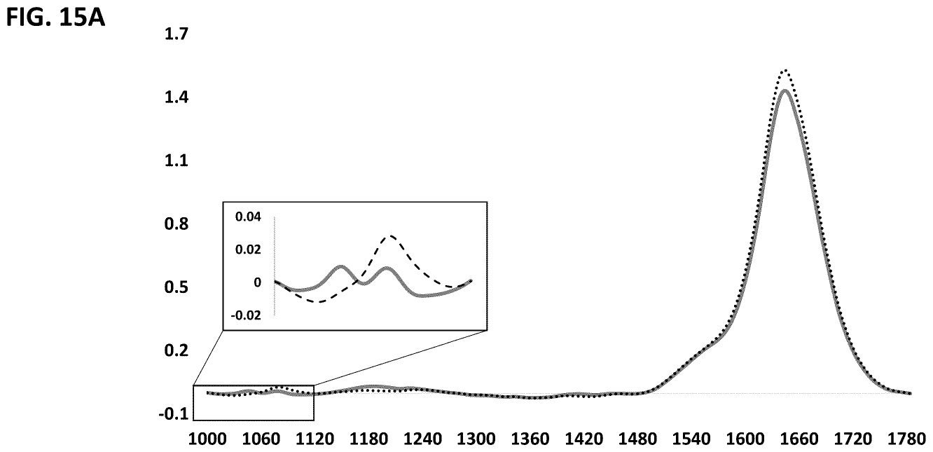

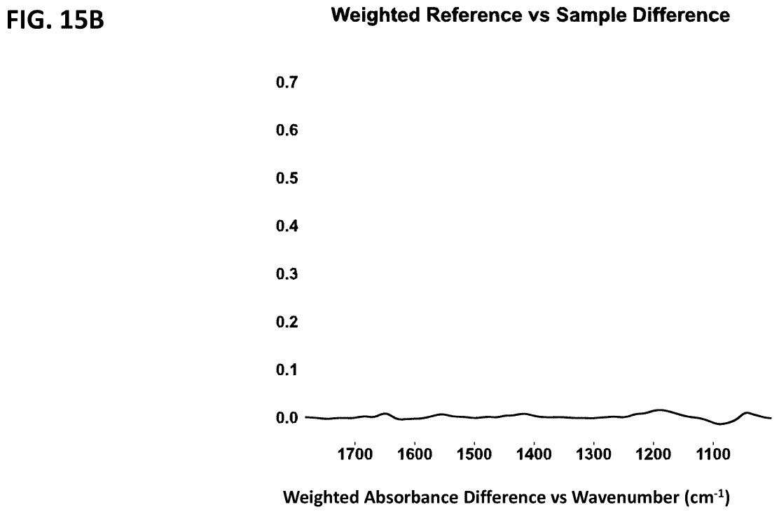

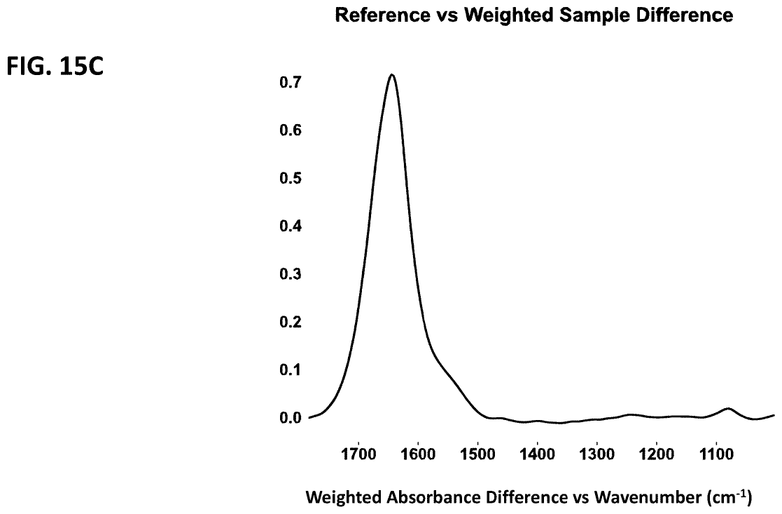

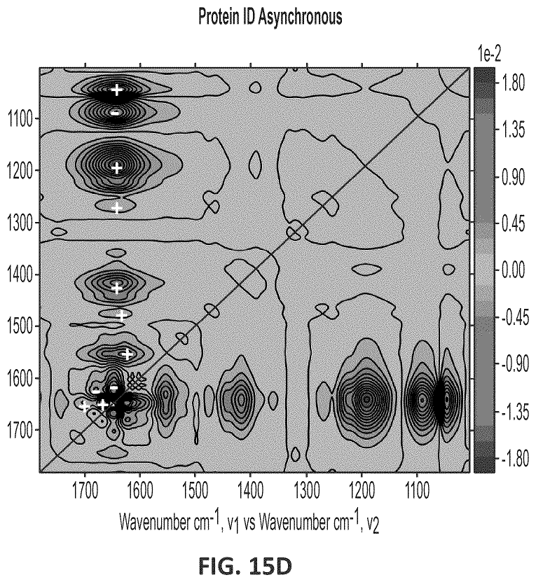

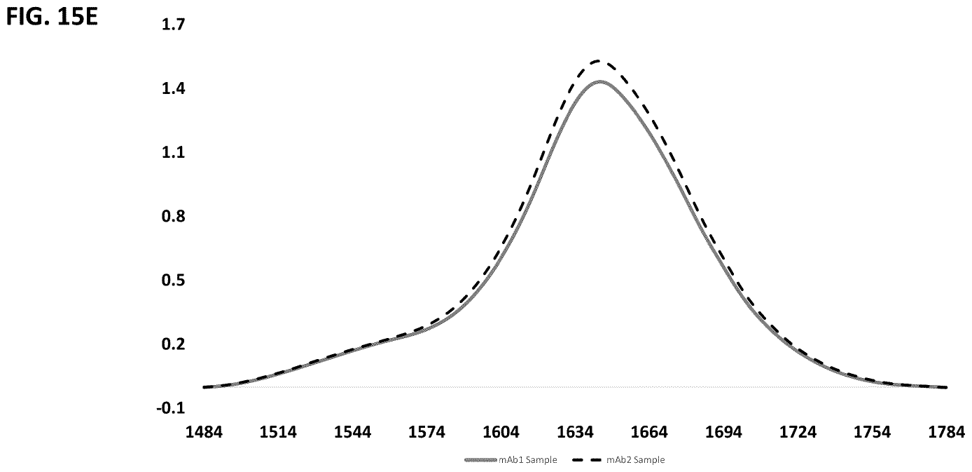

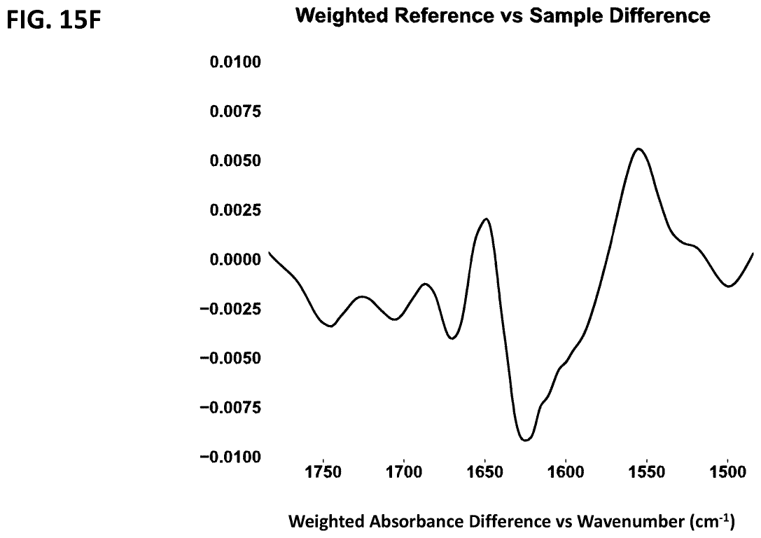

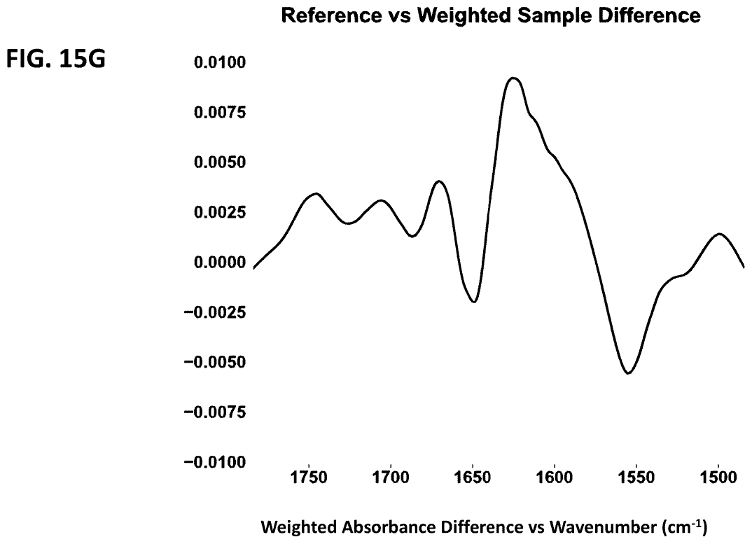

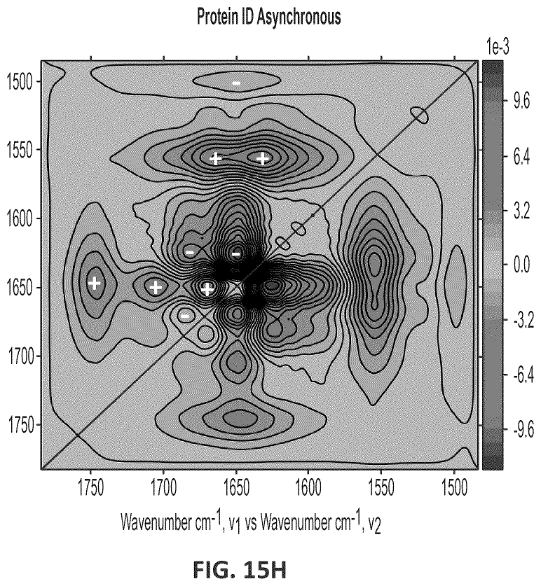

FIG. 15A shows an illustration of an mAb2 sample and a reference sample of mAb1 at room temperature within the spectral region 1780-1015 cm⁻¹ acquired using a QCLM. Differences in spectral contributions can be observed in the glycosylation spectral region of 1134-1015 cm⁻¹, establishing differences in glycan composition between the mAb2 sample and the mAb1 reference sample (as shown in the zoomed in, blow up insert in FIG. 15A). Also, intensity differences in the amide 1 band are shown between the mAb2 samples and the mAb1 reference sample. FIG. 15B is an illustration of a 2T2D weighted difference spectra for the reference sample of mAb1 within the full spectral region of 1780-1015 cm⁻¹. FIG. 15C is an illustration of a 2T2D weighted difference spectra for the sample of mAb2 within the full spectral region of 1780-1015 cm⁻¹. FIG. 15D is an illustration of the 2T2D asynchronous plot of the mAb2 sample when compared to the mAb1 reference full spectral region of 1780-1015 cm⁻¹. The cross peak assignments that aid in establishing the spectral differences are summarized in Tables V-A and V-B for the positive and negative cross peaks, respectively. The method can assess differences due to post-translational modification such as glycosylation when comparing against a reference standard, including by evaluating and examining the cross peaks. The extent of glycosylation of the desired protein may be evaluated as part of determining the identity of the protein. FIG. 15E shows an illustration of a spectral overlay of a mAb2 sample and a reference sample of mAb1 at room temperature within the spectral region 1780-1486 cm⁻¹. FIG. 15F is an illustration of a 2T2D weighted difference spectra for the reference sample of mAb1 within the amide I and II band spectral region of 1780-1486 cm⁻¹. FIG. 15G is an illustration of a 2T2D weighted difference spectra for the sample of mAb2 within the spectral region of 1780-1486 cm⁻¹. FIG. 15H is an illustration of the 2T2D asynchronous plot of the mAb2 sample when compared to the mAb1 reference within the amide I and II band spectral region of 1780-1486 cm⁻¹. The cross peaks and their assignments for the mAb2 sample and a reference sample of mAb1 are summarized in Tables V-C and V-D. In this case, mAb1 and mAb2 share the same IgG scaffold and have considerable sequence identity within the FAB regions which also include the complementary determining regions (CDRs). These highly homologous engineered therapeutic proteins served to prove the capability of the method in discerning protein identity. The differences in cross peaks often also provide actual amino acid differences within secondary structure type allowing the probing of the intact proteins in their formulation providing in-depth understanding during a streamline comparative approach.

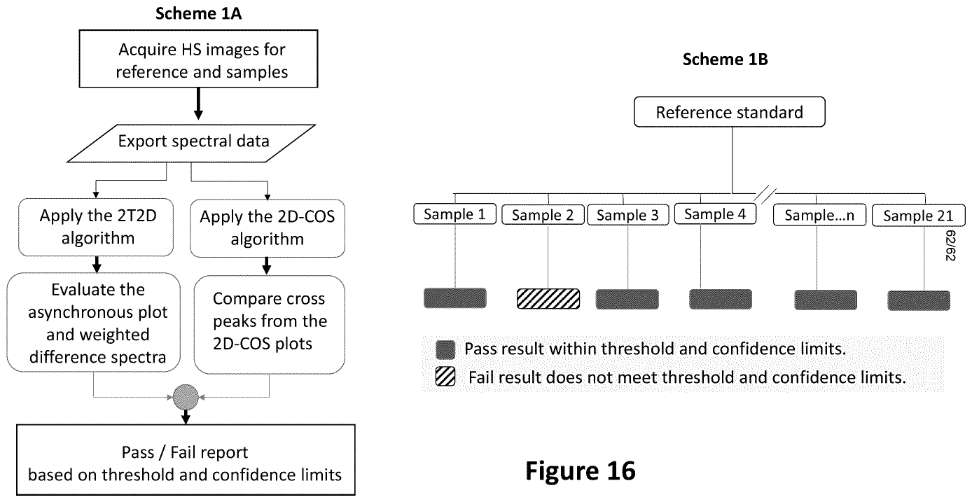

FIG. 16 is a schematic illustration of the protein identification and purity assessment workflow. As shown in Scheme 1A in FIG. 16, the assessment may begin with acquiring spectral images, such as hyperspectral images, of a reference standard sample and one or more samples to be evaluated. The spectral images may be, for example, hyperspectral images acquired with a QCLM. Spectral data for the sample and reference standard sample are acquired and exported to an automated evaluation process. The automated evaluation process may be performed using a computer, with instruction executed by a processor performing the evaluation process steps. The instructions may be included on one or more non-transitory, computer readable media. As part of the process, a 2T2D algorithm is applied to the spectral data for the sample and reference sample, and 2T2D synchronous plots, asynchronous plots, and weighted difference spectra are generated. The asynchronous plots and weighted difference spectra are then evaluated. The asynchronous plots may be evaluated to determine if cross peaks are present, indicating a difference between the identifies of the sample being evaluated and the reference sample. If cross peaks are present, then the number, position, and value of the cross peaks is evaluated against a predetermined threshold or confidence limit. If the evaluation shows the cross peaks are within the threshold or limit. The threshold or limit may vary depending on the protein or other product being evaluated. Similarly, the weighted difference spectra are evaluated and again a determination is made as to whether the difference is within a threshold or confidence limit. If the weighted difference is within the threshold or confidence limit, then the identity of the protein is confirmed as being that of the reference standard. In addition, a 2D-COS algorithm may be applied to the spectral data as part of the process, and 2D-COS synchronous and asynchronous plots may be generated. The cross peaks in the 2D-COS plots for the sample may be compared to those of the reference standard and evaluated to determine the purity of the protein sample as compared to the standard. As shown in Scheme 1B, the protein identity and purity assessment may be performed on a plurality of samples, such as 21 samples, at the same time. These samples may be compared to one or more reference samples of a particular protein, and pass/fail results of the identification process indicated for each of the samples. The process is not limited by modality, and may be applied to proteins, peptides, mAbs, bispecifics, different modalities including: antibody drug conjugates, for example. The process may also be applied to assess other biopharma samples, such as Adeno-associated viruses and lipid nanoparticles.

TABLE I
Summary of protein ID & purity comparability assessment for both spectral regions.

| | | | | Amide I and II bands spectral | Full spectral region of | |
|---|---|---|---|---|---|---|
| Application | Application | | | region of: 1780-1486 cm⁻¹ | 1780 - 1015 cm⁻¹ | Confirmed |
| Table # | FIG. # | Reference description | Sample description | Euclidian distance* | Euclidian distance* | identity |
| — | 11 (A-D) | mAb1 | mAb1 | | 0.00 | ✓ |
| — | 11 (E-G) | mAb1 | mAb1 | 0.00 | | |
| — | 12 (A-C) | glycosylated target | glycosylated target | | 0.00 | ✓ |
| — | 12 (D-F) | glycosylated target | glycosylated target | 0.00 | | |
| — | — | mAb2 | mAb2 | | 0.00 | ✓ |
| — | — | mAb2 | mAb2 | 0.00 | | |
| Table II (A, B) | 13 (A-D) | glycosylated target | mAb1 | | 6.52 | x |
| Table II (C-H) | 13 (E-L) | glycosylated target | mAb1 | 5.10 | | |
| Table III (A, B) | — | glycosylated target | mAb2 | | 8.36 | x |
| Table III (C-F) | — | glycosylated target | mAb2 | 9.17 | | |
| Table IV (A, B) | 14 (A-D) | glycosylated target | mAb2 / target complex | | 5.67 | x |
| | | | (2/1, mol ratio) | | | |
| Table IV (C-F) | 14 (E-J) | glycosylated target | mAb2 / target complex | 1.57 | | |
| | | | (2/1, mol ratio) | | | |
| Table V (A, B) | 15 (A-D) | mAb1 | mAb2 | | 5.51 | x |
| Table V (C, D) | 15 (E-H) | mAb1 | mAb2 | 0.06 | | |

*note:
values based on the weighted sample absorbance difference.

TABLE II-A
2T2D asynchronous summary of the mAb1 associated cross peaks that highlight the spectral contribution
differences between the glycosylated target and mAb1 within the full spectral region (1780-1015 cm⁻¹).

| Positive | | |
|---|---|---|
| cross peaks | Cross peak Assignments | Comment |
| (1685, 1600) | β-turn, His | Histidine located in a β-turn. |
| (1663, 1606) | Hinge loop, His | Histidine located in the hinge loop |
| (1643, 1612) | RC, Asn | asparagine located in the random coil |
| (1643, 1459) | RC, δCH₂ | methylene scissoring mode |
| (1643, 1432) | RC, β(CH) | CH in-plane bending |
| (1643, 1364) | RC, β(OH) | OH in-plane bending |
| (1643, 1336) | RC, ω(CH₂) | CH₂ wagging |
| (1643, 1147) | RC, νₐₛ(C—O—C, C—C) | asymmetric stretch (C—O—C, C—C) ring vibrations |
| (1643, 1105) | RC, ν(CC), ν(CO), β(COH) | galactose stretching (CC, CO) and bending (COH) vibrations |
| (1643, 1049) | RC, νₛ (C—O—C, C—C) | symmetric stretch (C—O—C, C—C) ring vibrations |
| (1643, 1034) | RC, ν(C—O—C, C—OH) | stretching (C—O—C, C—OH) glucose ether linkage and secondary OH |
| (1643, 1082) | RC, δ(C₁—H) | glucose bending vibration |

note:
Cross peaks observed within 1500-1015 cm⁻¹ are glycosylation associated vibrational modes.
note:
RC, random coil

TABLE II-B
2T2D asynchronous summary of the glycosylated target associated cross peaks
that highlight the spectral contribution differences between the glycosylated target
and mAb1 within the full spectral region (1780-1015 cm⁻¹).

| Negative | | |
|---|---|---|
| cross peaks | Cross peak Assignments | Comment |
| (1645, 1545) | α-helix, Glu- | glutamate located in a helix. |
| (1643, 1283) | RC, β(CH), β(CH₂) | CH and CH₂ in-plane bending vibration |
| (1643, 1260) | RC, β(CH), β(CH₂) | glucose CH and CH₂ in-plane bending |
| (1643, 1245) | RC, β(OH) | OH in-plane bending |
| (1643, 1220) | RC, τ(CH₂) | glucose CH₂ twisting vibration |
| (1643, 1180) | RC, νₐₛ(C—O—C, C—C) | asymmetric stretch (C—O—C, C—C) ring vibrations |

TABLE II-C
2T2D asynchronous summary of the mAb1 associated
cross peaks that highlight the spectral contribution
differences between the glycosylated target and mAb1 within
the spectral region 1780-1486 cm⁻¹.

| Positive | Cross peak | |
|---|---|---|
| cross peaks | Assignments | Comment |
| (1636, 1612) | β-sheet, Asn | Asparagine located in a β-sheet. |
| (1630, 1612) | Arg, Asn | Intra-molecular hydrogen bonding |
| | | interaction. |
| (1625, 1608) | Lys, His | Intra-molecular hydrogen bonding |
| | | interaction. |
| (1662, 1604) | loop, His | Histidine located in a hinge loop region. |
| (1636, 1625) | β-sheet, Lys | Lysine located in a β-sheet. |
| (1643, 1625) | RC, Lys | Arginine located in a random coil. |
| (1643, 1630) | RC, Arg | Lysine located in a random coil. |

TABLE II-D
2T2D asynchronous summary of the glycosylated target
associated cross peaks that highlight the spectral contribution
differences between the glycosilated target and mAb1 within
the spectral region 1780-1486 cm⁻¹.

| Negative | Cross peak | |
|---|---|---|
| cross peaks | Assignments | Comment |
| (1741, 1643) | Asp/Glu, RC | non-Hydrogen bonded |
| (1722, 1643) | Asp/Glu, RC | 2-Hydrogen-bonded carboxylic acid |
| (1712, 1643) | Asp/Glu, RC | 1-Hydrogen-bonded carboxylic acid |
| (1705, 1643) | Asp/Glu, RC | 1-Hydrogen-bonded carboxylic acid |
| (1645, 1545) | α-helix, Glu | Glutamate located in a helix. |
| (1645, 1534) | α-helix, Glu | Glutamate located in a helix. |
| (1645, 1525) | α-helix, Lys | Lysine located in a helix. |
| (1645, 1520) | α-helix, Tyr | Tyrosine located in a helix. |
| (1645, 1510) | α-helix, Phe | Phenylalanine located in a helix. |
| (1645, 1500) | α-helix, Trp | Tryptophan located in a helix. |
| (1695, 1576) | β-turn, Asp | Aspartate located in a β-turn. |
| (1695, 1581) | β-turn, Gln | Glutamine located in a β-turn. |
| (1709, 1518) | Asp/Glu, Tyr | 2-Hydrogen-bonded carboxylic acid |
| (1721, 1518) | Asp/Glu, Tyr | 1-Hydrogen-bonded carboxylic acid |
| (1712, 1554) | Asp/Glu, Glu- | 1-Hydrogen-bonded carboxylic acid & |
| | | Glu- |
| (1705, 1554) | Asp/Glu, Glu- | 1-Hydrogen-bonded carboxylic acid & |
| | | Glu- |
| (1720, 1554) | Asp/Glu, Glu- | 1-Hydrogen-bonded carboxylic acid & |
| | | Glu- |
| (1733, 1554) | Asp/Glu, Glu- | 1-Hydrogen-bonded carboxylic acid & |
| | | Glu- |

TABLE II-E
2D-COS synchronous plot summary of mAb1 associated cross
peaks during the temperature range of 24-52° C.

| | | Cross peak | |
|---|---|---|---|
| Cross peaks | Sign | Assignments | Comment |
| (1758, 1636) | + | Asp/Glu, β-sheet | Asp/Glu non-Hydrogen bonded perturbed within a β-sheet. |
| (1695, 1636) | − | β-turn, β-sheet | correlation identifies parallel β-sheets are perturbed |
| (1636, 1554) | − | β-sheet, Glu- | Glutamate perturbed within a β-sheet. |
| (1636, 1534) | − | β-sheet, Glu- | Glutamate perturbed within a β-sheet. |

TABLE II-F
2D-COS asynchronous plot summary of mAb1 associated
cross peaks during the temperature range of 24-52° C.

| Cross peaks | Sign | Cross peak Assignments | Comment |
|---|---|---|---|
| (1760, 1695) | + | Asp/Glu, β-turn | Asp/Glu non-Hydrogen bonded perturbed in a β-turn. |
| (1760, 1663) | + | Asp/Glu, loop | Asp/Glu non-Hydrogen bonded perturbed in a loop. |
| (1727, 1695) | + | Asp/Glu, β-turn | Asp/Glu 2-Hydrogen bonded perturbed within a β-turn. |
| (1727, 1712) | + | Asp/Glu, Asp/Glu | Asp/Glu 2- and 1-Hydrogen-bonded carboxylic acid side chain. |
| (1745, 1636) | + | Asp/Glu, β-sheet | Asp/Glu non-Hydrogen bonded perturbed in a β-sheet. |
| (1695, 1545) | + | β-turn, Glu- | Glutamate perturbed within a β-turn. |
| (1663, 1534) | + | hinge loop, Glu- | Glutamate perturbed within a hinge loop. |
| (1636, 1500) | + | β-sheet, Trp | Tryptophan perturbed within a β-sheet. |
| (1636, 1572) | + | β-sheet, Asp- | Aspartate perturbed within a β-sheet. |
| (1645, 1630) | + | α-helix, Arg | Arginine perturbed within a helix. |
| (1525, 1518) | + | Lys, Tyr | Lysine-tyrosine hydrogen bond disruption |
| (1760, 1608) | − | Asp/Glu, His | Asp/Glu non-Hydrogen bonded and a neighboring His are perturbed. |
| (1760, 1643) | − | Asp/Glu, RC | Asp/Glu non-Hydrogen bonded perturbed in a random coil. |
| (1745, 1708) | − | Asp/Glu, Asp/Glu | Asp/Glu 1- and non-Hydrogen bonded perturbed. |
| (1745, 1663) | − | Asp/Glu, loop | Asp/Glu non-Hydrogen bonded perturbed in a hinge loop. |
| (1721, 1636) | − | Asp/Glu, β-sheet | 2-Hydrogen-bonded carboxylic acid perurbed within a β-sheet. |
| (1695, 1653) | − | β-turn, π-helix | β-turn is perturbed after the π-helix. |
| (1688, 1636) | − | β-turn, β-sheet | β-turn is perturbed after the β-sheet. |
| (1663, 1636) | − | hinge loop, β-sheet | hinge loop is perturbed after the β-sheet. |
| (1643, 1518) | − | RC, Tyr | The random coil is perturbed after the tyrosines. |
| (1630, 1612) | − | Arg, Asn | Arginine is perturbed after the asparagine. |
| (1625, 1545) | − | Lys, Glu- | Lysine - glutamate salt-bridge disruption. |
| (1608, 1545) | − | His, Glu- | Histidine is perturbed after the glutamate. |
| (1608, 1518) | − | His, Tyr | Histidine - tyrosine hydrogen bonding disruption. |

TABLE II-G
2D-COS synchronous plot summary of pure glycosylated target associated
cross peaks during the temperature range of 24-52° C.

| | | Cross peak | |
|---|---|---|---|
| Cross peaks | Sign | Assignments | Comment |
| (1758, 1636) | + | Asp/Glu, β-sheet | Asp/Glu non-Hydrogen bonded perturbed within a β-sheet. |
| (1695, 1636) | − | β-turn, β-sheet | correlation identifies parallel β-sheets are perturbed |
| (1636, 1554) | − | β-sheet, Glu- | Glutamate perturbed within a β-sheet. |
| (1636, 1534) | − | β-sheet, Glu- | Glutamate perturbed within a β-sheet. |

TABLE II-H
2D-COS asynchronous plot summary of pure glycosylated target associated
cross peaks during the temperature range of 24-52° C.

| | | Cross peak | |
|---|---|---|---|
| Cross peaks | Sign | Assignments | Comment |
| (1760, 1695) | + | Asp/Glu, β-turn | Asp/Glu non-Hydrogen bonded perturbed in a β-turn. |
| (1760, 1672) | + | Asp/Glu, Asn | Asp/Glu non-Hydrogen bonded are perturbed prior to asparagine. |
| (1745, 1636) | + | Asp/Glu, β-sheet | Asp/Glu non-Hydrogen bonded perturbed in a β-sheet. |
| (1695, 1680) | + | β-turn, β-turn | β-turn (1695 cm⁻¹) is perturbed prior to β-turn (1680 cm⁻¹). |
| (1695, 1545) | + | β-turn, Glu- | Glutamate perturbed within a β-turn. |
| (1695, 1518) | + | β-turn, Tyr | Tyrosine perturbed within a β-turn. |
| (1672, 1534) | + | Asn, Glu- | Asparagine is perturbed prior to glutamate. |
| (1663, 1525) | + | hinge loop, Lys | Lysine is perturbed within a hinge loop. |
| (1643, 1630) | + | RC, Arg | The random coil is perturbed prior to arginines. |
| (1636, 1600) | + | β-sheet, His | Histidine is perturbed within a β-sheet. |
| (1636, 1572) | + | β-sheet, Asp- | Aspartate perturbed within a β-sheet. |
| (1572, 1545) | + | Asp-, Glu- | Aspartates are perturbed prior to glutamates. |
| (1525, 1518) | + | Lys, Tyr | Lysine-tyrosine hydrogen bond disruption |
| (1760, 1643) | − | Asp/Glu, RC | Asp/Glu non-Hydrogen bonded perturbed in a random coil. |
| (1745, 1695) | − | Asp/Glu, β-turn | Asp/Glu non-Hydrogen bonded perturbed in a β-turn. |
| (1745, 1672) | − | Asp/Glu, Asn | Asp/Glu non-Hydrogen bonded perturbed after asparagine. |
| (1721, 1636) | − | Asp/Glu, β-sheet | 2-Hydrogen-bonded carboxylic acid perurbed within a β-sheet. |
| (1712, 1695) | − | Asp/Glu, β-turn | 2-Hydrogen-bonded carboxylic acid perurbed within a β-turn. |
| (1712, 1581) | − | Asp/Glu, Gln | 2-Hydrogen-bonded carboxylic acid is perurbed after glutamine. |
| (1695, 1653) | − | β-turn, π-helix | β-turn is perturbed after the π-helix. |
| (1688, 1636) | − | β-turn, β-sheet | β-turn is perturbed after the β-sheet. |
| (1680, 1581) | − | β-turn, Gln | Glutamine perturbed within a β-turn. |
| (1663, 1636) | − | hinge loop, β-sheet | hinge loop is perturbed after the β-sheet. |
| (1643, 1518) | − | RC, Tyr | The random coil is perturbed after the tyrosines. |
| (1636, 1545) | − | β-sheet, Glu- | Glutamate perturbed within a β-sheet. |
| (1612, 1518) | − | Asn, Tyr | Asparagine -tyrosine hydrogen bond disruption. |
| (1608, 1545) | − | His, Glu- | Histidine is perturbed after the glutamate. |

TABLE III-A
2T2D asynchronous summary of the mAb2 associated cross peaks that highlight the spectral contribution
differences between the glycosylated target and mAb2 within the full spectral region (1780-1015 cm⁻¹).

| Positive | | |
|---|---|---|
| cross peaks | Cross peak assignments | Comment |
| (1636, 1612) | β-sheet, Asn | Asparagine located in a β-sheet. |
| (1643, 1460) | RC, δCH₂ | methylene scissoring mode |
| (1643, 1429) | RC, β(CH) | CH in-plane bending |
| (1567, 1368) | ν(COO—), β(OH) | carboxylate stretching & OH in-plane bending |
| (1643, 1364) | RC, β(OH) | OH in-plane bending |
| (1572, 1342) | ν(COO—), β(CH), β(CH₂) | carboxylate stretching & CH and CH₂ in-plane bending |
| (1643, 1342) | RC, β(CH), β(CH₂) | CH and CH₂ in-plane bending |
| (1643, 1147) | RC, νₐₛ(C—O—C, C—C) | asymmetric stretch (C—O—C, C—C) ring vibrations |
| (1643, 1105) | RC, ν(CC), ν(CO), β(COH) | galactose stretching (CC, CO) and bending (COH) vibrations |
| (1643, 1034) | RC, ν(C—O—C, C—OH) | stretching (C—O—C, C—OH) glucose ether linkage and secondary OH |

TABLE III-B
2T2D asynchronous summary of the glycosylated target associated cross peaks
that highlight the spectral contribution differences between the glycosylated target
and mAb2 within the full spectral region (1780-1015 cm⁻¹).

| Negative | | |
|---|---|---|
| cross peaks | Cross peak assignments | Comment |
| (1727, 1636) | Asp/Glu, β-sheet | 2-Hydrogen-bonded carboxylic acid |
| (1645, 1545) | α-helix, Glu- | glutamate located in a helix. |
| (1643, 1245) | RC, β(OH) | OH in-plane bending |
| (1643, 1220) | RC, τ(CH₂) | glucose CH₂ twisting vibration |
| (1643, 1183) | RC, νₐₛ(C—O—C, C—C) | asymmetric stretch (C—O—C, C—C) ring vibrations |
| (1643, 1080) | RC, δ(C₁—H) | glucose bending vibration |
| (1643, 1070) | RC, νₛ(C—O—C, C—C) | symmetric stretch (C—O—C, C—C) ring vibrations |

TABLE III-C
2T2D asynchronous summary of the mAb2 associated
cross peaks that highlight the spectral contribution
differences between the glycosylated target and mAb2 within
the spectral region 1780-1486 cm⁻¹.

| Positive | Cross peak | |
|---|---|---|
| cross peaks | Assignments | Comment |
| (1636, 1612) | β-sheet, Asn | Asparagine located in a β-sheet. |
| (1636, 1601) | β-sheet, His | Histidine located in a β-sheet. |
| (1625, 1612) | Lys, Asn | Intra-molecular hydrogen bonding |
| | | interaction. |
| (1625, 1604) | Lys, His | Intra-molecular hydrogen bonding |
| | | interaction. |

TABLE III-D
2T2D asynchronous summary of the glycosylated target
associated cross peaks that highlight the spectral contribution
differences between the glycosylated target and mAb2 within
the spectral region 1780-1486 cm⁻¹.

| Negative | Cross peak | |
|---|---|---|
| cross peaks | Assignments | Comment |
| (1727, 1636) | Asp/Glu, β-sheet | 2-Hydrogen-bonded carboxylic acid |
| (1712, 1636) | Asp/Glu, β-sheet | 1-Hydrogen-bonded carboxylic acid |
| (1705, 1636) | Asp/Glu, β-sheet | 1-Hydrogen-bonded carboxylic acid |
| (1705, 1663) | Asp/Glu, hinge | 1-Hydrogen-bonded carboxylic acid |
| | loop | |
| (1712, 1545) | Asp/Glu, Glu- | 1-Hydrogen-bonded carboxylic acid |
| | | & Glu- |
| (1653, 1630) | π-helix, Arg | Arginine located in a π-helix. |
| (1653, 1625) | π-helix, Lys | Lysine located in a π-helix. |
| (1653, 1612) | π-helix, Asn | Asparagine located in a π-helix. |
| (1685, 1576) | β-turn, Asp | Aspartate located in a β-turn. |
| (1685, 1581) | β-turn, Gln | Glutamine located in a β-turn. |
| (1646, 1545) | α-helix, Glu | Glutamate located in a helix. |
| (1636, 1545) | β-sheet, Glu | Glutamate located in a β-sheet. |

TABLE III-E
2D-COS synchronous plot summary of mAb2 associated cross
peaks during the temperature range of 24-52° C.

| | | Cross peak | |
|---|---|---|---|
| Cross peaks | Sign | Assignments | Comment |
| (1760, 1636) | + | Asp/Glu, β-sheet | Asp/Glu non-Hydrogen bonded perturbed within a β-sheet. |
| (1695, 1554) | + | β-turn, Glu- | Glutamate perturbed within a β-turn. |
| (1695, 1534) | + | β-turn, Glu- | Glutamate perturbed within a β-turn. |
| (1636, 1581) | − | β-sheet, Gln | Glutamine perturbed within a β-sheet. |
| (1636, 1554) | − | β-sheet, Glu- | Glutamate perturbed within a β-sheet. |
| (1636, 1534) | − | β-sheet, Glu- | Glutamate perturbed within a β-sheet. |

TABLE III-F
2D-COS asynchronous plot summary of mAb2 associated
cross peaks during the temperature range of 24-52° C.

| | | Cross peak | |
|---|---|---|---|
| Cross peaks | Sign | Assignments | Comment |
| (1760, 1695) | + | Asp/Glu, β-turn | Asp/Glu non-Hydrogen bonded perturbed in a β-turn. |
| (1760, 1672) | + | Asp/Glu, Asn | Asp/Glu non-Hydrogen bonded are perturbed prior to asparagine. |
| (1745, 1636) | + | Asp/Glu, β-sheet | Asp/Glu non-Hydrogen bonded perturbed in a β-sheet. |
| (1695, 1545) | + | β-turn, Glu- | Glutamate perturbed within a β-turn. |
| (1695, 1518) | + | β-turn, Tyr | Tyrosine perturbed within a β-turn. |
| (1672, 1545) | + | Asn, Glu- | Asparagine is perturbed prior to glutamate. |
| (1663, 1534) | + | hinge loop, Glu- | Glutamate is perturbed within a hinge loop. |
| (1663, 1518) | + | hinge loop, Tyr | Tyrosine is perturbed within a hinge loop. |
| (1643, 1630) | + | RC, Arg | The random coil is perturbed prior to arginines. |
| (1636, 1572) | + | β-sheet, Asp- | Aspartate perturbed within a β-sheet. |
| (1572, 1545) | + | Asp-, Glu- | Aspartates are perturbed prior to glutamates. |
| (1760, 1636) | − | Asp/Glu, α-helix | Asp/Glu non-Hydrogen bonded perturbed in a helix. |
| (1745, 1695) | − | Asp/Glu, β-turn | Asp/Glu non-Hydrogen bonded perturbed in a β-turn. |
| (1745, 1545) | − | Asp/Glu, Glu- | Asp/Glu non-Hydrogen bonded perturbed after glutamate. |
| (1721, 1636) | − | Asp/Glu, β-sheet | 2-Hydrogen-bonded carboxylic acid perurbed within a β-sheet. |
| (1712, 1636) | − | Asp/Glu, β-sheet | 2-Hydrogen-bonded carboxylic acid perurbed within a β-sheet. |
| (1712, 1545) | − | Asp/Glu, Glu- | 2-Hydrogen-bonded carboxylic acid is perurbed after glutamate. |
| (1695, 1636) | − | β-turn, α-helix | β-turn is perturbed after the α-helix. |
| (1695, 1660) | − | β-turn, hinge loop | β-turn is perturbed after the hinge loop. |
| (1695, 1572) | − | β-turn, Asp- | Aspartate located in a β-turn. |
| (1676, 1572) | − | Arg, Asp- | Arginine aspartate salt-bridge disruption |
| (1663, 1636) | − | hinge loop, β-sheet | hinge loop is perturbed after the β-sheet. |
| (1636, 1545) | − | β-sheet, Glu- | Glutamate perturbed within a β-sheet. |
| (1636, 1518) | − | β-sheet, Tyr | Tyrosine perturbed within a β-sheet. |
| (1608, 1545) | − | His, Glu- | Histidine is perturbed after the glutamate. |
| (1545, 1504) | − | Glu-, Phe | Glutamate is perturbed after the phenylalanine. |

TABLE IV-A
2T2D asynchronous summary of the glycosylated target/mAb2 (2.1, mol ratio) mixture associated cross
peaks that highlight the spectral contribution differences between the pure glycosylated target and target/mAb2
(2.1, mol ratio) complex within the full spectral region (1780-1015 cm⁻¹).

| Positive | | |
|---|---|---|
| cross peaks | Cross peak Assignments | Comment |
| (1643, 1600) | RC, His | Histidine located in a random coil. |
| (1636, 1600) | β-sheet, His | Histidine located in a β-sheet. |
| (1645, 1432) | α-helix, β(CH) | CH in-plane bending (for an aa side chain) |
| (1695, 1432) | β-turn, β(CH) | CH in-plane bending (for an aa side chain) |
| (1643, 1365) | RC, β(OH) | OH in-plane bending |
| (1643, 1338) | RC, β(CH), β(CH₂) | CH and CH₂ in-plane bending |
| (1643, 1105) | RC, ν(CC), ν(CO), β(COH) | galactose stretching (CC, CO) and bending (COH) vibrations |
| (1643, 1034) | RC, ν(C—O—C, C—OH) | stretching (C—O—C, C—OH) glucose ether linkage and secondary OH |

TABLE IV-B
2T2D asynchronous summary of the pure glycosylated target associated cross peaks that
highlight the spectral contribution differences between the glycosylated target and target/mAb2
(2.1, mol ratio) mixture within the full spectral region (1780-1015 cm⁻¹).

| Negative | | |
|---|---|---|
| cross peaks | Cross peak Assignments | Comment |
| (1705, 1663) | Asp/Glu, hinge loop | 1-Hydrogen-bonded carboxylic acid |
| (1705, 1636) | Asp/Glu, β-sheet | 1-Hydrogen-bonded carboxylic acid |
| (1643, 1545) | RC, ν(COO—) | carboxylate stretching |
| (1643, 1492) | RC, δCH₂ | galactose, methylene scissoring mode |
| (1643, 1478) | RC, δCH₂ | methylene scissoring mode |
| (1643, 1292) | RC, τ(CH₂) | glucose CH2 twisting vibration |
| (1643, 1282) | RC, τ(CH₂) | galactose CH₂ twisting vibration |
| (1643, 1254) | RC, β(CH), β(CH₂) | glucose CH and CH₂ in-plane bending |
| (1643, 1245) | RC, β(OH) | OH in-plane bending |
| (1643, 1222) | RC, τ(CH₂) | glucose CH₂ twisting vibration |
| (1643, 1180) | RC, δ(C₁—H) | glucose bending vibration |
| (1643, 1132) | RC, ν(CO), ν(CC) | glucose stretching (CC, CO) |
| (1643, 1090) | RC, ν(CC), ν(CO), β(COH) | galactose stretching (CC, CO) and bending (COH) vibrations |
| (1643, 1070) | RC, ν(C—O—C, C—OH) | stretching (C—O—C, C—OH) glucose ether linkage and secondary OH |

TABLE IV-C
2T2D asynchronous summary of the target/mAb2 (2.1, mol
ratio) complex associated cross peaks that highlight
the spectral contribution differences between the glycosylated
target and target/mAb2 (2.1, mol ratio) mixture within the
spectral region 1780-1486 cm⁻¹.

| Positive | Cross peak | |
|---|---|---|
| cross peaks | Assignments | Comment |
| (1636, 1630) | β-sheet, Arg | Arginine located in a β-sheet. |
| (1636, 1625) | β-sheet, Lys | Lysine located in a β-sheet. |
| (1636, 1612) | β-sheet, Asn | Asparagine located in a β-sheet. |
| (1636, 1604) | β-sheet, His | Histidine located in a β-sheet. |
| (1636, 1600) | β-sheet, His | Histidine located in a β-sheet. |
| (1636, 1581) | β-sheet, Gln | Glutamine located in a β-sheet. |
| (1630, 1604) | Arg, His | Intra-molecular hydrogen bonding |
| | | interaction. |
| (1625, 1604) | Lys, His | Intra-molecular hydrogen bonding |
| | | interaction. |
| (1643, 1630) | RC, Arg | Arginine located in a random coil. |
| (1643, 1625) | RC, Lys | Lysine located in a random coil. |
| (1643, 1600) | RC, His | Histidine located in a random coil. |
| (1646, 1600) | α-helix, His | Histidine located in a helix. |
| (1646, 1581) | α-helix, Gln | Glutamine located in a helix. |
| (1746, 1742) | Asp, Glu | non-Hydrogen bonded |
| (1754, 1742) | Asp, Glu | non-Hydrogen bonded |
| (1754, 1727) | Asp, Glu | non-Hydrogen bonded & 2-H-bonded |
| | | carboxylic acid |

TABLE IV-D
2T2D asynchronous summary of the glycosylated target associated cross peaks that
highlight the spectral contribution differences between the glycosylated target
and target/mAb2 (2.1, mol ratio) mixture within the spectral region 1780-1486 cm⁻¹.

| Negative | | |
|---|---|---|
| cross peaks | Cross peak Assignments | Comment |
| (1734, 1636) | Asp/Glu, β-sheet | non-Hydrogen bonded |
| (1727, 1636) | Asp/Glu, β-sheet | 2-Hydrogen-bonded carboxylic acid |
| (1712, 1636) | Asp/Glu, β-sheet | 1-Hydrogen-bonded carboxylic acid |
| (1705, 1636) | Asp/Glu, β-sheet | 1-Hydrogen-bonded carboxylic acid |
| (1734, 1643) | Asp/Glu, RC | non-Hydrogen bonded |
| (1722, 1643) | Asp/Glu, RC | 2-Hydrogen-bonded carboxylic acid |
| (1712, 1643) | Asp/Glu, RC | 1-Hydrogen-bonded carboxylic acid |
| (1705, 1643) | Asp/Glu, RC | 1-Hydrogen-bonded carboxylic acid |
| (1705, 1663) | Asp/Glu, hinge loop | 1-Hydrogen-bonded carboxylic acid |
| (1712, 1554) | Asp/Glu, Glu- | 1-Hydrogen-bonded carboxylic acid & Glu- |
| (1721, 1518) | Asp/Glu, Tyr | 1-Hydrogen-bonded carboxylic acid |
| (1643, 1500) | RC, Trp | Tryptophan located in a random coil. |
| (1643, 1504) | RC, Phe | Phenylalanine located in a random coil. |
| (1643, 1518) | RC, Tyr | Tyrosine located in a random coil. |
| (1643, 1525) | RC, Lys | Lysine located in a random coil. |
| (1680, 1600) | β-turn, His | Histidine located in a β-turn. |
| (1685, 1545) | β-turn, Glu | Glutamate located in a β-turn. |
| (1660, 1545) | hinge loop, Glu | Glutamate located in a hinge loop. |
| (1695, 1545) | β-turn, Glu | Glutamate located in a β-turn. |
| (1685, 1672) | β-turn, Asn | Asparagine located in a β-turn. |
| (1685, 1676) | β-turn, Arg | Arginine located in a β-turn. |

TABLE IV-E
2D-COS synchronous plot summary of the glycosylated target/mAb2 (2.1, mol ratio)
mixture associated cross peaks during the temperature range of 24-52° C.

| Cross peaks | Sign | Cross peak Assignments | Comment |
|---|---|---|---|
| (1760, 1636) | + | Asp/Glu, β-sheet | Asp/Glu non-Hydrogen bonded perturbed within a β-sheet. |
| (1695, 1567) | + | β-turn, Asp- | Aspartate perturbed within a β-turn. |
| (1695, 1554) | + | β-turn, Glu- | Glutamate perturbed within a β-turn. |
| (1695, 1534) | + | β-turn, Glu- | Glutamate perturbed within a β-turn. |
| (1676, 1554) | + | Arg, Glu | salt-bridge interaction |
| (1676, 1534) | + | Arg, Glu | salt-bridge interaction |
| (1676, 1636) | − | Arg, β-sheet | Arginine perturbed within a β-sheet. |
| (1695, 1636) | − | β-turn, β-sheet | β-turn is perturbed after the β-sheet. |
| (1636, 1581) | − | β-sheet, Gln | Glutamine perturbed within a β-sheet. |
| (1636, 1567) | − | β-sheet, Asp- | Aspartate perturbed within a β-sheet. |
| (1636, 1554) | − | β-sheet, Glu- | Glutamate perturbed within a β-sheet. |
| (1636, 1534) | − | β-sheet, Glu- | Glutamate perturbed within a β-sheet. |

TABLE IV-F
2D-COS asynchronous plot summary of the glycosylated target/mAb2 (2.1, mol ratio)
mixture associated cross peaks during the temperature range of 24-52° C.

| Cross peaks | Sign | Cross peak Assignments | Comment |
|---|---|---|---|
| (1760, 1695) | + | Asp/Glu, β-turn | Asp/Glu non-Hydrogen bonded perturbed in a β-turn. |
| (1760, 1663) | + | Asp/Glu, hinge loop | Asp/Glu non-Hydrogen bonded are perturbed in the hinge loop. |
| (1745, 1636) | + | Asp/Glu, β-sheet | Asp/Glu non-Hydrogen bonded perturbed in a β-sheet. |
| (1721, 1695) | + | Asp/Glu, β-turn | 2-Hydrogen-bonded carboxylic acid perurbed within a β-turn. |
| (1695, 1545) | + | β-turn, Glu- | Glutamate perturbed within a β-turn. |
| (1695, 1518) | + | β-turn, Tyr | Tyrosine perturbed within a β-turn. |
| (1663, 1545) | + | hinge loop, Glu- | Glutamate perturbed within a hinge loop. |
| (1663, 1534) | + | hinge loop, Glu- | Glutamate perturbed within a hinge loop. |
| (1663, 1518) | + | hinge loop, Tyr | Tyrosine perturbed within a hinge loop. |
| (1646, 1630) | + | α-helix, Arg | Arginine located in an α-helix. |
| (1636, 1572) | + | β-sheet, Asp- | Aspartate perturbed within a β-sheet. |
| (1625, 1600) | + | Lys, His | Lysine-histidine hydrogen bonding disruption. |
| (1572, 1545) | + | Asp-, Glu- | Aspartates are perturbed prior to glutamates. |
| (1572, 1518) | + | Asp-, Tyr | Aspartates are perturbed prior to tyrosine. |
| (1534, 1518) | + | Glu-, Tyr | Glutamates are perturbed prior to tyrosine. |
| (1534, 1504) | + | Glu-, Phe | Glutamates are perturbed prior to phenylalanine. |
| (1504, 1500) | + | Tyr, Trp | Tyrosines are perturbed prior to tryptophans. |
| (1760, 1645) | − | Asp/Glu, α-helix | Asp/Glu non-Hydrogen bonded are perturbed in the helix. |
| (1745, 1695) | − | Asp/Glu, β-turn | Asp/Glu non-Hydrogen bonded perturbed in a β-turn. |
| (1745, 1545) | − | Asp/Glu, Glu- | Asp/Glu non-Hydrogen bonded perturbed after glutamate. |
| (1721, 1636) | − | Asp/Glu, β-sheet | 2-Hydrogen-bonded carboxylic acid perurbed within a β-sheet. |
| (1712, 1688) | − | Asp/Glu, β-turn | 2-Hydrogen-bonded carboxylic acid perurbed within a β-turn. |
| (1712, 1575) | − | Asp/Glu, Asp- | 2-Hydrogen-bonded carboxylic acid is perurbed after aspartate. |
| (1695, 1660) | − | β-turn, hinge loop | β-turn is perturbed after the hinge loop. |
| (1695, 1636) | − | β-turn, α-helix | β-turn is perturbed after the α-helix. |
| (1695, 1572) | − | β-turn, Asp- | Aspartate located in a β-turn. |
| (1676, 1572) | − | Arg, Asp- | Arginine aspartate salt-bridge disruption |
| (1636, 1545) | − | β-sheet, Glu- | Glutamate perturbed within a β-sheet. |
| (1636, 1518) | − | β-sheet, Tyr | Tyrosine perturbed within a β-sheet. |
| (1608, 1545) | − | His, Glu- | Histidine is perturbed after the glutamate. |
| (1608, 1504) | − | His, Phe | Histidine is perturbed after the phenylalanine. |
| (1545, 1504) | − | Glu-, Phe | Glutamate is perturbed after the phenylalanine. |

TABLE V-A
2T2D asynchronous summary of the mAb1 associated cross peaks that highlight the spectral contribution
differences between mAb1 and mAb2 within the full spectral region (1780-1015 cm⁻¹).

| Positive | | |
|---|---|---|
| cross peaks | Cross peak assignments | Comment |
| (1737, 1653) | Asp/Glu, π-helix | Asp/Glu in a non-H-bonded form located in a π-helix. |
| (1705, 1653) | Asp/Glu, π-helix | Asp/Glu located in a π-helix in the 1-H-bonded carboxylic acid form. |
| (1672, 1653) | Asn, π-helix | Asparagine located in a π-helix. |
| (1630, 1554) | Arg, Glu | salt-bridge interaction |
| (1663, 1554) | hinge loop, Glu- | Glutamate located in a hinge loop. |
| (1695, 1554) | β-turn, Glu- | Glutamate located in a β-turn. |
| (1705, 1554) | Glu, Glu- | Asp in a 1-H-bonded carboxylic acid form & aspartate |
| (1663, 1525) | hinge loop, Lys | Lysine located in a hinge loop. |
| (1645, 1432) | α-helix, β(CH) | CH in-plane bending (for an aa side chain) |
| (1643, 1478) | RC, δCH₂ | methylene scissoring mode |
| (1643, 1386) | RC, ω(CH₂) | glucose CH₂ wagging |
| (1643, 1360) | RC, β(OH) | OH in-plane bending |
| (1643, 1211) | RC, τ(CH₂) | glucose CH2 twisting vibration |
| (1643, 1190) | RC, δ(C₁—H) | glucose bending vibration |
| (1643, 1034) | RC, ν(C—O—C, C—OH) | stretching (C—O—C, C—OH) glucose ether linkage and secondary OH |

TABLE V-B
2T2D asynchronous summary of the mAb2 associated cross peaks that highlight the spectral contribution
differences between mAb2 and mAb1 within the full spectral region (1780-1015 cm⁻¹).

| Negative | | |
|---|---|---|
| cross peaks | Cross peak assignments | Comment |
| (1721, 1625) | Asp/Glu, Lys | 2-Hydrogen-bonded carboxylic acid & Lys |
| (1721, 1600) | Asp/Glu, His | 2-Hydrogen-bonded carboxylic acid & His |
| (1685, 1672) | β-turn, Asn | Asparagine located in a β-turn. |
| (1653, 1625) | π-helix, Lys | Lysine located in a π-helix. |
| (1680, 1625) | β-turn, Lys | Lysine located in a β-turn. |
| (1680, 1612) | β-turn, Asn | Asparagine located in a β-turn. |
| (1680, 1600) | β-turn, His | Histidine located in a β-turn. |
| (1643, 1085) | RC, ν(CC), ν(CO), β(COH) | galactose stretching (CC, CO) and bending (COH) vibrations |

TABLE V-C
2T2D asynchronous summary of the mAb1 associated cross peaks that highlight the spectral
contribution differences between mAb1 and mAb2 within the spectral region 1780-1486 cm⁻¹.

| Positive | | |
|---|---|---|
| cross peaks | Cross peak Assignments | Comment |
| (1672, 1653) | Asn, π-helix | Asparagine located in a π-helix. |
| (1705, 1653) | Asp/Glu, π-helix | Asp/Glu located in a π-helix in the 1-H-bonded carboxylic acid form. |
| (1705, 1660) | Asp/Glu, hinge loop | Asp/Glu located in a loop in the 1-H-bonded carboxylic acid form. |
| (1705, 1685) | Asp/Glu, β-turn | Asp/Glu located in a β-turn in the 1-H-bonded carboxylic acid form. |
| (1734, 1653) | Asp/Glu, π-helix | Asp/Glu in a non-H-bonded form located in a π-helix. |
| (1746, 1696) | Asp/Glu, β-turn | Asp/Glu in a non-H-bonded form located in a β-turn. |
| (1746, 1660) | Asp/Glu, hinge loop | Asp/Glu in a non-H-bonded form located in a hinge loop. |
| (1708, 1567) | Asp, Asp- | Asp in a 1-H-bonded carboxylic acid form |
| (1712, 1554) | Glu, Glu- | Glu in a 1-H-bonded carboxylic acid form |
| (1727, 1554) | Asp/Glu, Glu- | 2-Hydrogen-bonded carboxylic acid |
| (1630, 1554) | Arg, Glu | salt-bridge interaction |
| (1685, 1554) | β-turn, Glu | Glutamate located in a β-turn. |
| (1695, 1554) | β-turn, Glu | Glutamate located in a β-turn. |
| (1612, 1554) | Asn, Glu | Intra-molecular hydrogen bonding interaction. |
| (1625, 1534) | Lys, Glu | salt-bridge interaction |
| (1625, 1518) | Lys, Tyr | Intra-molecular hydrogen bonding interaction. |

TABLE V-D
2T2D asynchronous summary of the mAb2 associated cross peaks
that highlight the spectral contribution differences between mAb2
and mAb1 within the spectral region 1780-1486 cm⁻¹.

| Negative | | |
|---|---|---|
| cross peaks | Cross peak Assignments | Comment |
| (1712, 1600) | Asp/Glu, His | 1-Hydrogen-bonded carboxylic acid & His |
| (1708, 1608) | Asp, His | Asp in a 1-H-bonded carboxylic acid form & His |
| (1653, 1625) | π-helix, Lys | Lysine located in a π-helix. |
| (1653, 1612) | π-helix, Asn | Asparagine located in a π-helix. |
| (1653, 1604) | π-helix, His | Histidine located in a π-helix. |
| (1646, 1581) | α-helix, Gln | Glutamine located in an α-helix. |
| (1685, 1581) | β-turn, Gln | Glutamine located in a β-turn. |
| (1685, 1672) | β-turn, Asn | Asparagine located in a β-turn. |
| (1680, 1612) | β-turn, Asn | Asparagine located in a β-turn. |
| (1680, 1600) | β-turn, His | Histadine located in a β-turn. |
| (1680, 1625) | β-turn, Lys | Lysine located in a β-turn. |
| (1646, 1504) | α-helix, Phe | Phenylalanine located in an α-helix. |
| (1646, 1496) | α-helix, Trp | Tryptophan located in an α-helix. |
| (1676, 1496) | Arg, Trp | Intra-molecular hydrogen bonding interaction. |
| (1625, 1496) | Lys, Trp | Intra-molecular hydrogen bonding interaction. |

1. A method for performing protein identification screening of a sample comprising:
a) providing the sample in a slide containing at least one sample well and at least one reference well;
b) acquiring at least one spectral image of the sample and at least one spectral image of the at least one reference using a quantum cascade laser microscope under controlled temperature conditions;
c) identifying and selecting, in at least one of the acquired first spectral images, a region of interest;
d) obtaining spectral data for the sample and the at least one reference for the region of interest;
e) applying a baseline correction to the spectral data for the sample and the reference for the region of interest;
f) applying a two-trace two-dimensional correlation to the baseline corrected sample and reference spectral data to generate a synchronous spectrum Φ(v₁, v₂) and asynchronous spectrum Ψ(v₁, v₂);
g) generating weighted difference spectra for the sample and reference; and
h) analyzing the weighted difference spectra and confirming an identity of the sample protein when the weighted difference is within a predetermined threshold.

2. The method of claim 1, wherein acquiring at least one spectral image of the sample using a quantum cascade laser microscope includes acquiring at least one first hyperspectral image of the sample.

3. The method of claim 1, wherein generating a synchronous spectrum Φ(v₁, v₂) and asynchronous spectrum Ψ(v₁, v₂) comprising generating a two-trace two-dimensional synchronous plot and a two-trace two-dimensional asynchronous plot.

4. The method of claim 3, further comprising identifying at least one cross peak in the two-trace two-dimensional asynchronous plot, and analyzing the at least one cross peak to determine glycan composition differences between the sample and the reference.

5. The method of claim 3, further comprising identifying at least one cross peak in the two-trace two-dimensional asynchronous plot, and analyzing the at least one cross peak to determine differences in amino acid composition within a secondary structure type.

6. The method of claim 3, further comprising identifying at least one cross peak in the two-trace two-dimensional asynchronous plot, and analyzing the at least one cross peak to determine differences in amino acid composition within a secondary structure type.

7. The method of claim 3, further comprising identifying at least one cross peak in the two-trace two-dimensional asynchronous plot, and analyzing the at least one cross peak to determine differences in weak interactions.

8. The method of claim 3, further comprising identifying at least one cross peak in the two-trace two-dimensional asynchronous plot, and analyzing the at least one cross peak to determine differences in amino acid composition within a secondary structure type.

9. The method of claim 3, further comprising identifying at least one cross peak in the two-trace two-dimensional asynchronous plot, and analyzing the at least one cross peak to determine a presence of degradation products in the sample, the degradation products being at least one of deamidation, oxidation, or aggregates.

10. The method of claim 3, further comprising generating a plurality of two-trace two-dimensional synchronous plots and two-trace two-dimensional asynchronous plots for the sample and reference over time, and analyzing the stability and dynamics of the sample by comparing the two-trace two-dimensional synchronous plots and two-trace two-dimensional asynchronous plots for the sample to the two-trace two-dimensional synchronous plots and two-trace two-dimensional asynchronous plots for the reference.

11. The method of claim 1, further comprising:
applying two-dimensional correlation (2DCOS) analysis to generate a synchronous correlation plot and an asynchronous correlation plot for the sample;
identifying in the synchronous correlation plot positive cross peaks that correlate with auto peaks associated with aggregation of proteins, peptides, and/or new modalities in the sample; and
using identified peak intensities of the spectral data to determine an amount of aggregation of proteins, peptides, and/or new modalities in the sample.

12. A system for performing protein identification screening of a sample comprising:
a slide containing at least one sample well and at least one reference well;
a data acquisition module, including a quantum cascade laser microscope, configured to:
acquire at least one spectral image of the sample and at least one spectral image of the at least one reference under controlled temperature conditions;
identify and select, in at least one of the acquired first spectral images, a region of interest;
obtain spectral data for the sample and the at least one reference for the region of interest;
apply a baseline correction to the spectral data for the sample and the reference for the region of interest;
apply a two-trace two-dimensional correlation to the baseline corrected sample and reference spectral data to generate a synchronous spectrum Φ(v₁, v₂) and asynchronous spectrum Ψ(v₁, v₂);
generate weighted difference spectra for the sample and reference; and
analyze the weighted difference spectra and confirming an identity of the sample protein when the weighted difference is within a predetermined threshold.
